SEQUENCE LISTING (11 sequences)

SEQ ID NO: 1 - protein, 19 aa, Artificial sequence
  c-Myc peptide sequence (51-69 positions)
KKFELLPTPPLSPSRRSGL

SEQ ID NO: 2 - protein, 399 aa, Artificial sequence
  GSK3beta fragment amino acid sequence GSK 35-433
SKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKDSSGTGHFTSGVRVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 3 - DNA, 1197 bp, Artificial sequence
  GSK3beta fragment nucleotide sequence GSK 35-433
agcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggattcgtcaggaacaggacatttcacctcaggagtgcgggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacc

SEQ ID NO: 4 - DNA, 1194 bp, Artificial sequence
  Firefly luciferase fragment nucleotide sequence NFL-398 (1-398)
atggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagagctgcctgcgtcagattctcgcatgccagagatcctatttttggcaatcaaatcgctccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcctggccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccaagggggatgataaaccgggcgcggtcggtaaagttgttccattttttgaagcgaaggttgtggatctggataccgggaaaacgctgggcgttaatcagagaggcgaattatgtgtcagaggacctatgattatg

SEQ ID NO: 5 - protein, 398 aa, Artificial sequence
  Firefly luciferase N-terminal fragment amino acid sequence NFL-398 (1-398)
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRAACVRFSHARDPIFGNQIAPDTAILSVVPFHHGFGMFTTLGYLICGFRVVLMYRFEEELFLRSLQDYKIQSALLVPTLFSFLAKSTLIDKYDLSNLHEIASGGAPLSKEVGEAVAKRFHLPGIRQGYGLTETTSAILITPKGDDKPGAVGKVVPFFEAKVVDLDTGKTLGVNQRGELCVRGPMIM

SEQ ID NO: 6 - DNA, 471 bp, Artificial sequence
  Firefly luciferase C-terminal fragment nucleotide sequence CFL (394-550)
cctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 7 - protein, 156 aa, Artificial sequence
  Firefly luciferase C-terminal fragment amino acid sequence CFL (394-550)
PMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 8 - protein, 433 aa, Artificial sequence
  GSK3beta full-length amino acid sequence
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKDSSGTGHFTSGVRVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 9 - protein, 417 aa, Artificial sequence
  First polypeptide having luciferase N-terminal region NFL(1-398) and c-Myc region
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRAACVRFSHARDPIFGNQIAPDTAILSVVPFHHGFGMFTTLGYLICGFRVVLMYRFEEELFLRSLQDYKIQSALLVPTLFSFLAKSTLIDKYDLSNLHEIASGGAPLSKEVGEAVAKRFHLPGIRQGYGLTETTSAILITPKGDDKPGAVGKVVPFFEAKVVDLDTGKTLGVNQRGELCVRGPMIMKKFELLPTPPLSPSRRSGL

SEQ ID NO: 10 - protein, 555 aa, Artificial sequence
  Second polypeptide sequence havng a GSK3beta region and the C-region of luciferase (CFL(394-550))
SKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKDSSGTGHFTSGVRVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNSTPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 11 - DNA, 60 bp, Artificial sequence
  Nucleotide sequence encoding region 51-69 of c-Myc
aaaaagtttgaattattacctacacccccgttatcaccaagtaggagaagcggattataa

(19) United States
(12) Patent Application Publication
(10) Pub. No.: US 2012/0270914 A1
(43) Pub. Date: Oct. 25, 2012

(54) SPLIT-LUCIFERASE C-MYC SENSOR AND USES THEREOF

(75) Inventors: Hua Fan-Minogue, Milpitas, CA (US); Sanjiv S. Gambhir, Portola Valley, CA (US)

(73) Assignee: THE BOARD OF TRUSTEES OF THE LELAND STANFORD JUNIOR UNIVERSITY, PALO ALTO, CA (US)

(21) Appl. No.: 13/455,521

(22) Filed: Apr. 25, 2012

Related U.S. Application Data
(60) Provisional application No. 61/478,571, filed on Apr. 25, 2011.

(51) Int. Cl. C12Q 1/66; C12N 5/09; C12N 5/10; C12N 5/071; A61K 31/426; C12N 15/85

(52) U.S. Cl. 514/371; 435/8; 435/320.1; 435/325; 435/366; 435/375

(57) ABSTRACT

A split luciferase-based sensor system was developed to noninvasively monitor and image phosphorylation-mediated c-Myc activation, in which the complementation of the split FL is induced by phosphorylation-mediated interaction between GSK3β and c-Myc. The complemented luciferase activity resulting from this interaction is specific to c-Myc phosphorylation and correlated with the steady-state and temporal regulation of c-Myc phosphorylation in cell culture. The sensor system also allows monitoring of c-Myc—targeted drug efficacy in intact cells and living animals. This new imaging sensor can provide insight into the role of functional c-Myc in cancer biology and is useful for the discovery and development of specific anti-c-Myc drugs.

### CROSS-REFERENCE TO RELATED APPLICATION

This application claims priority from U.S. Patent Application Ser. No. 61/478,571 entitled “SPLIT-LUCIFERASE C-MYC SENSOR AND USES THEREOF” filed on Apr. 25, 2011, the entirety of which is hereby incorporated by reference.

### STATEMENT REGARDING FEDERALLY SPONSORED RESEARCH OR DEVELOPMENT

This invention was made with government support under NIH Grant Nos.: CA082214 and CA118681 awarded by the U.S. National Institutes of Health of the United States government. The government has certain rights in the invention

### TECHNICAL FIELD

The present disclosure is generally related to a system for the detection of Myc activation, and its uses in imaging Myc activation and as an assay for modulators thereof. The present disclosure further relates to high-throughput assays for the screening of compounds useful as modulators of Myc activation.

### BACKGROUND

The Myc gene encodes transcription factors (N-Myc, c-Myc, and L-Myc) that regulate up to 15% of all vertebrate genes, essential to almost every aspect of cell behavior, including cell growth and proliferation, cell cycle progression, differentiation, and apoptosis (Dang et al. (2006) Semin Cancer Biol 16: 253-264). The c-Myc protein in particular coordinates the integration of extracellular and intracellular signals as the central hub for cellular cues (Sodir & Evan (2009) J. Biol. 8: 77). In light of these functions, it is not surprising that expression of c-Myc is tightly regulated in normal cells. Normally, cells exhibit low steady-state levels of c-Myc expression when in a non-proliferative state. In the presence of stimulatory signals, such as developmental cues or mitogens, c-Myc is phosphorylated at Ser-62 (S62) through Ras-induced ERK pathway activation (Sears et al. (2000) Genes Dev. 14: 2501-2514), which temporarily activates and stabilizes the protein. On removal of the stimuli, phosphorylated S62 is recognized by glycogen synthase kinase-3β (GSK3β), which further phosphorylates Thr-58 (T58) and leads to ubiquitination and rapid proteasomal degradation (Yeh et al. (2004) Nat. Cell. Biol. 6: 308-318). The phosphorylation-mediated temporary c-Myc activation is essential for many cellular processes, including entry into cell cycle phases, biogenesis of ribosomes, response to oxidative stress, and induction of apoptosis (Hann (2006) Semin. Cancer Biol. 16: 288-302).

The tight control of c-Myc activity is defective at multiple levels in almost all human cancers, where the protein is constitutively activated and stabilized. This also makes c-Myc an attractive candidate for targeted cancer therapy (Vita & Henriksson (2006) Semin. Cancer Biol. 16: 318-330). Current strategies are aimed mainly at down-regulating c-Myc by inhibiting gene expression, such as using antisense oligonucleotides and RNAi to compete for binding to the c-Myc promoter, its coding region, or downstream target genes (Wang et al. (2005) Breast Cancer Res. 7: R220-228; Kimura et al., (1995) Cancer Res. 55: 1379-1384; Kim & Miller (1995) Biochemistry 34: 8165-8171). Although these approaches can inhibit tumor growth and promote apoptosis, the main disadvantages are the instability of the short oligonucleotides used and the difficulty of in vivo delivery (Vita & Henriksson (2006) Semin. Cancer Biol. 16: 318-330). Some attempts to repress c-Myc at the protein level (e.g., the use of small molecules to disrupt c-Myc interaction) have shown promise in cell culture (Berg et al., (2002) Proc. Natl. Acad. Sci. U.S.A. 99: 3830-3835; Mo & Henriksson (2006) Proc. Natl. Acad. Sci. U.S.A. 103: 6344-6349). To date, approaches to regulating phosphorylation-mediated c-Myc activity, which is essential for sustaining the growth of many tumors (Hann (2006) Semin. Cancer Biol. 16: 288-302), have been limited. ERK kinase inhibitors PD98059 and U0126 decrease the c-Myc phosphorylation level in vitro (Hydbring et al,. (2009) Proc. Natl. Acad. Sci. U.S.A. 107: 58-63), but there has been no study of their effect on tumor growth. Atorvastatin (AT), a member of the statin family, was unexpectedly found to reduce phosphorylation of c-Myc by inhibiting 3-hydroxy-3-methylglutaryl-coenzyme A (HMG-coA) reductase and in turn preventing c-Myc—induced lymphomagenesis (Shachaf et al., (2007) Blood 110: 2674-2684), although the exact molecular mechanism remains unclear. The unavailability of methods to non-invasively monitor c-Myc activity has hindered further understanding of Myc cancer biology and contributes to delays in c-Myc—targeted drug development (Meyer & Penn (2008) Nat. Rev. Cancer 8: 976-990).

Multimodality molecular imaging has emerged as a key spectrum of technologies to advance the understanding of disease mechanisms and accelerate drug discovery and development (Willmann et al., (2008) Nat. Rev. Drug Discov. 7: 591-607). In particular, reporter gene imaging strategies based on protein-assisted complementation of split luciferases are emerging as powerful tools for detecting and quantifying induced protein interactions and functional protein modifications in vivo, such as ubiquitination and phosphorylation (Kaihara et al., (2003) Anal. Chem.75: 4176-4181; Luker et al., (2003) Nat. Med. 9: 969-973; Chan et al. (2008) Cancer Res. 68: 216-226; Paulmurugan & Gambhir (2007) Anal. Chem. 79: 2346-2353).

### SUMMARY

Briefly described, one aspect of the present disclosure encompasses embodiments of a system for detecting the activation of a Myc peptide, the system comprising: a first polypeptide comprising a region isolated from a Myc polypeptide having at least one phosphor site associated with Myc activation and is resistant to ubiquitin-mediated proteosomal degradation, where said region is covalently linked to a first fragment of a luciferase; and a second polypeptide comprising a region of a glycogen synthase kinase 3β (GSK3β) capable of selectively interacting with a phosphorylated region of a Myc polypeptide, and a second fragment of the luciferase, where the region isolated from Myc polypeptide, when phosphorylated, can selectively bind to the glycogen synthase kinase 3β (GSK3β) region of the second polypeptide, allowing the first and the second luciferase fragments to cooperatively interact to produce a detectable signal.

In embodiments of this aspect of the disclosure, the region of a glycogen synthase kinase 3β (GSK3β) can extend from about amino acid position 35 to about amino acid position 433 of the amino acid sequence according to SEQ ID NO.: 8.

In embodiments of this aspect of the disclosure, the region isolated from Myc polypeptide can comprise the sequence according to SEQ ID NO.: 1, or a conservative derivative thereof.

In embodiments of this aspect of the disclosure, the region of the glycogen synthase kinase 3β (GSK3β) polypeptide can comprise the sequence according to SEQ ID NO.: 2, or a conservative derivative thereof.

In embodiments of this aspect of the disclosure, the first polypeptide can have an amino acid sequence having at least 90% similarity to the amino acid sequence according to SEQ ID NO.: 9.

In embodiments of this aspect of the disclosure, the second polypeptide can have an amino acid sequence having at least 90% similarity to the amino acid sequence according to SEQ ID NO.: 10.

In embodiments of this aspect of the disclosure, the system can further comprise a genetically modified animal or human cell where the first and the second polypeptides can be expressed from at least one heterologous nucleic acid of the genetically modified animal or human cell.

In embodiments of this aspect of the disclosure, the genetically modified animal or human cell can respond to an exogenous ligand by phosphorylating the Myc polypeptide or region thereof, stimulating the association of the Myc polypeptide or region thereof and the glycogen synthase kinase 3β (GSK3β) region, allowing the first and the second fragments of the luciferase to cooperatively associate to generate a detectable signal.

Another aspect of the disclosure encompasses embodiments of a recombinant nucleic acid system comprising: (a) a first expression cassette comprising a first nucleotide sequence encoding a region of a Myc polypeptide resistant to ubiquitin-mediated proteosomal degradation and a first region of a luciferase, and where the first nucleotide sequence is operably linked to a promoter region for expressing the region of the Myc polypeptide and the first region of the luciferase as a single polypeptide; and (b) a second expression cassette comprising a second nucleotide sequence encoding a region of a glycogen synthase kinase 3β (GSK3β) and a second region of the luciferase, and where the second nucleotide sequence is operably linked to a second promoter region for expressing the region of a glycogen synthase kinase 3β (GSK3β) and the first region of the luciferase as a single polypeptide.

In embodiments of this aspect of the disclosure, the region of the glycogen synthase kinase 3β (GSK3β) can extend from amino acid position 35 to about position 433 of the amino acid sequence according to SEQ ID NO.: 8,

In embodiments of this aspect of the disclosure, the first and the second expression cassettes can each be in separate expression vectors, or are in the same expression vector.

In embodiments of this aspect of the disclosure, the region of a Myc polypeptide resistant to ubiquitin-mediated proteosomal degradation can be encoded by a nucleic acid sequence having at least 90% similarity to the sequence according to SEQ ID No.: 11; the first region of a luciferase can be encoded by a nucleotide sequence having at least 90% similarity to the sequence according to SEQ ID No.: 6, the region of a glycogen synthase kinase 3β (GSK3β) can be encoded by a nucleotide sequence having at least 90% similarity to the sequence according to SEQ ID No.: 3, and the second region of the luciferase can be encoded by a nucleotide sequence having at least 90% similarity to the sequence according to SEQ ID No.: 4.

In some embodiments of this aspect of the disclosure, the region of a Myc polypeptide resistant to ubiquitin-mediated proteosomal degradation can be encoded by a nucleic acid sequence according to SEQ ID No.: 11; the first region of a luciferase can be encoded by a nucleotide sequence according to SEQ ID No.: 6, the region of a glycogen synthase kinase 3β (GSK3β) can be encoded by a nucleotide sequence according to SEQ ID No.: 3, and the second region of the luciferase can be encoded by a nucleotide sequence according to SEQ ID No.: 4.

In embodiments of this aspect of the disclosure, the system can be within an animal or human cell.

Yet another aspect of the disclosure encompasses genetically modified animal or human cell or a population of genetically modified animal or human cells comprising a recombinant nucleic acid system according to any of the aforementioned embodiments.

Still another aspect of the disclosure encompasses embodiments of a method of detecting Myc activation in a population of animal or human cells, the method comprising the steps of: (i) providing a genetically modified population of animal or human cells comprising a recombinant nucleic acid system comprising: (a) a first expression cassette comprising a first nucleotide sequence encoding a region of a Myc polypeptide resistant to ubiquitin-mediated proteosomal degradation and a first region of a luciferase, and wherein the first nucleotide sequence is operably linked to a promoter region for expressing the region of the Myc polypeptide and the first region of the luciferase as a single polypeptide; and (b) a second expression cassette comprising a second nucleotide sequence encoding a region of a glycogen synthase kinase 3β (GSK3β) and a second region of the luciferase, and wherein the second nucleotide sequence is operably linked to a second promoter region for expressing the region of a glycogen synthase kinase 3β (GSK3β) and the first region of the luciferase as a single polypeptide; (ii) allowing the genetically modified population of animal or human cells to express the first and the second expression cassettes; (iii) contacting said cells with an agent characterized as stimulating the activation of a Myc polypeptide, thereby allowing the region of Myc polypeptide and the region of a glycogen synthase kinase 3β (GSK3β) of the expression products of the first and second cassettes to selectively bind to each other, and thereby allowing the first and the second fragments of the luciferase to cooperatively associate to produce a detectable signal; and (iv) detecting said signal, thereby detecting Myc activation in the cells. In embodiments of this aspect of the disclosure, the method can further comprise the step of generating an image of the distribution of the signal in the cells.

In embodiments of this aspect of the disclosure, the genetically modified animal or human cell can be an in vitro cultured animal or human cell.

In embodiments of this aspect of the disclosure, the genetically modified population of animal or human cells can be in a recipient animal or human.

In embodiments of this aspect of the disclosure, the genetically modified population of animal or human cells can be in a recipient animal or human and the image of the distribution of the signal in the genetically modified population of animal or human cells further provides an image of the distribution of Myc activation in the animal or human.

In embodiments of this aspect of the disclosure, the method can further comprise the steps of: (v) detecting quantitatively a first signal, thereby determining a first level of Myc activation in the genetically modified population of animal or human cells; (vi) contacting the genetically modified population of animal or human cells with an agent suspected of modulating the activation of Myc and detecting quantitatively a second signal, thereby determining a second level of Myc activation in the genetically modified population of animal or human cells; and (vii) comparing the first and the second levels of Myc activation, thereby determining if the agent modulates Myc activation.

In embodiments of this aspect of the disclosure, the agent can selectively bind to a receptor of the genetically modified population of animal or human cells, thereby modulating a signaling pathway that activates Myc.

In embodiments of this aspect of the disclosure, the method can configured as a high-throughput assay system for the screening of a plurality of agents suspected of modulating Myc activation in a cell.

Yet another aspect of the disclosure encompasses embodiments of a method of inhibiting the activation of Myc by a cell, comprising contacting the cell with an effective amount of a nitazoxanide, or a derivative thereof, thereby reducing the activation of Myc.

In embodiments of this aspect of the disclosure, the cell can be a cancer cell and inhibiting the activation of Myc can reduce the proliferation of the cancer cell.

Still yet another aspect of the disclosure encompasses embodiments of a composition comprising a therapeutic dose of nitazoxanide or a derivative thereof, and a pharmaceutically acceptable carrier.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIGS. 1A-1C illustrate the validation of a sensor system for imaging of c-Myc activation.

FIG. 1A shows a schematic strategy of imaging c-Myc activation by detecting S62 phosphorylation-mediated GSK3βc-Myc interaction. GSK3β and c-Myc fragments were fused, respectively, with C-terminal and N-terminal parts of inactive split Firefly luciferases. Upon growth signals, mediated Ras activation, c-Myc is phosphorylated at S62, which induces the recognition by the priming phosphate site of GSK3β. Further phosphorylation by the active site of GSK3β at T58 brings the split luciferase regions into closer proximity and complementation of bioluminescence activity.

FIG. 1B shows a diagram of different split luciferase fusion constructs. Different truncations of GSK3β and the c-Myc activation motif (51-69aa) were fused respectively with the C-terminal and N-terminal fragment of split luciferases as indicated. Numbers indicate the location of the amino acid in the sequence of full length GSK3β. The two phosphorylation sites, S62 and T58, are indicated.

FIG. 1C is a graph showing BLI of single and paired fusion constructs. NFL-SH2-SH2 is constructed by replacing the c-Myc motif with two SH2 domains of P13K. **, P<0.01 by unpaired t test.

FIGS. 2A and 2B illustrate the optimization of the sensor constructs.

FIG. 2A is a graph showing a comparison between complemented RL activity and complemented FL activity. Split RL fusion and split FL fusion of different truncations of GSK3β were co-expressed with split RL fusion and split FL fusion of the c-Myc activation motif respectively and subjected to BLI. *, P<0.05 by unpaired t test.

FIG. 2B is a graph showing BLI of S62 phosphorylation specificity of different truncations of GSK3β. Wild type, S62A and S62D mutants of NFL-c-Myc were co-expressed with CFL fused different truncation of GSK3β as indicated and subjected to BLI. *, P<0.05 by unpaired t test. NS, no significant difference.

FIGS. 3A-3C illustrate the characterization of the phosphorylation dependence of the complementation.

FIG. 3A is a diagram of the sensor system. Mutations at T58 and S62 are indicated.

FIG. 3B is a graph showing BLI of split FL complementation of NFL-c-Myc mutants and GSK 35-433-CFL. Wild type and mutant NFL-c-Myc as indicated were co-expressed with GSK 35-433-CFL and subjected to BLI. *, P<0.05, **, P<0.01 by unpaired t test.

FIG. 3C is a digital image of a Western blot analysis of cell lysates using phospho T58/S62 c-Myc antibody, firefly luciferase antibody and β-actin antibody.

FIGS. 4A-4C illustrate the detection of differential c-Myc phosphorylation level in normal and cancer cells.

FIG. 4A shows a digital image (top) of fluorescence produced when the sensor system is transfected in the indicated cell lines and subjected to BLI. The fluorescence activities were graphically shown (below). Complemented FL activity was normalized to co-expressed RL activity and plotted against drug concentrations in logarithmic scale.

FIG. 4B is a digital image of a Western blot analysis of the indicated cells using phospho T58/S62 c-Myc, c-Myc and β-actin antibody.

FIG. 4C is a graph showing the correlation coefficient between the c-Myc phosphorylation level and the normalized FL activity of each type of cells. R²=0.90.

FIGS. 5A-5C illustrate the detection of the temporal activation of c-Myc upon serum stimulation in cells.

FIG. 5A is a graph showing CHO cells, transfected with the sensor system, and serum starved for 24 hrs. They were then serum stimulated for the indicated time and subjected to BLI.

FIG. 5B is a digital image of a Western blot analysis of CHO cells upon serum stimulation for the indicated time using phospho c-Myc, c-Myc protein and β-actin antibodies.

FIG. 5C is a graph showing of correlation coefficient between the fold change of c-Myc phosphorylation level and the fold changed of FL activity at each time point. R²=0.87.

FIGS. 6A and 6B illustrate the detection of inhibition of c-Myc phosphorylation in intact cells.

FIG. 6A is a graph showing SKBR3 and 293T stable cells, constitutively expressing the c-Myc sensor (SK ST and 293T ST) or the full-length FL (SK FST and 293T FST) treated with indicated drugs at indicated concentration and subjected to BLI.

FIG. 6B is a graph showing a plot of correlation coefficient between the fold change of c-Myc phosphorylation level and the fold change of FL activity at different concentration of indicated drugs. Each R² is indicated.

FIGS. 7A-7E illustrate bioluminescence imaging of c-Myc phosphorylation in living mice.

FIG. 7A shows a diagram of the implanting location of the SKBR3 cells transiently transfected with combinations of plasmids as indicated.

FIG. 7B shows digital images of mice subjected to RL imaging with coelenterazine (Clz) and FL imaging with D-Luciferin (D-Luc). Autoluminescent signal of coelenterazine was seen in the liver (left).

FIG. 7C is a graph showing photon output for the complemented FL activity normalized to that for the RL activity (FL/RL) and plotted as the fold change to the FL/RL of the vector.

FIG. 7D shows digital images of Eμ-tTA/Tet-O-MYC transgenic mice (N=2) under AT or PBS treatment subjected to RL imaging with coelenterazine (Clz) and FL imaging with D-Luciferin (D-Luc) after hydrodynamic injection.

FIG. 7E is a digital image of a Western blot analysis of the liver tissue samples using phospho c-Myc, c-Myc and α-tubulin antibodies and the HE staining of the samples.

FIGS. 8A-8C illustrate bioluminescence imaging of AT inhibition of c-Myc phosphorylation in living mice.

FIG. 8A illustrates SKBR3 stable cells, constitutively expressing the c-Myc sensor (SK ST) or the full-length FL (SK FST), subcutaneously implanted as indicated and treated with AT or PBS. Representative images of FL imaging at indicated days of treatment are shown.

FIG. 8B shows a pair of graphs showing photon output of SK ST cells with AT or PBS treatment plotted together to show the AT inhibitory effect on the complemented FL activity (FIG. 8B, left). Photon output of SK FST cells with AT or PBS treatment were plotted together to show the AT inhibitory effect on the full-length FL activity (FIG. 8B, right).

FIG. 8C is a digital image of a Western blot analysis of xenograft tissue samples using phospho c-Myc, c-Myc and α-tubulin antibodies. Representative blots were shown.

FIG. 9 is a graph illustrating different S62 and T58 mutations introduced into NFL-c-Myc and co-expressed with GSK 35-433-CFL in SKBR3 cells. Cells were imaged with D-Luc in the IVIS 50 BLI system at 24 h after transfection. Cell lysates were collected for total protein determination.

FIGS. 10A-10D illustrate the phosphorylation sensor detecting the inhibitory effect of PD98059 in intact cells.

FIG. 10A is a digital image showing SKBR3 cells stably expressing the c-Myc phosphorylation sensor (SK ST) or the full-length FL (SK FST) treated with PD98059 at 0 μM, 10 μM, 20 μM, 50 μM, 100 μM, and 200 μM for 2 h. Cells were imaged with D-Luc in the IVIS 50 BLI system.

FIG. 10B is a graph showing the FL activities of sensor ST and FL ST cells normalized to the total protein content measured in each cell lysate. The normalized FL activities of cells treated with AT and cells treated without AT were compared to obtain the fold change.

FIG. 10C is a digital image of a Western blot analysis of the endogenous level of phospho T58/S62 of c-Myc, c-Myc protein, and β-actin on PD98059 treatment.

FIG. 10D is a graph illustrating the fold change of FL activity was correlated with the fold change of the c-Myc phosphorylation level (R2=0.91).

FIGS. 11A-11D illustrate the phosphorylation sensor detected the inhibitory effect of U0126 in intact cells.

FIG. 11A is a digital image of SkBR3 cells stably expressing the c-Myc phosphorylation sensor (SK ST) or the full-length FL (SK FST) treated with U0126 at 0 μM, 5 μM, 10 μM, 20 μM, 40 μM, and 80 μM for 2 h. Cells were imaged with D-Luc in the IVIS 50 BLI system.

FIG. 11B is a graph illustrating the FL activities of sensor ST and FL ST cells normalized to the total protein content measured from each cell lysate. The normalized FL activities of cells treated with AT and cells treated without AT were compared to obtain the fold change.

FIG. 11C is a digital image of a Western blot analysis of the endogenous level of phospho T58/S62 of c-Myc, c-Myc protein, and β-actin on U0126 treatment.

FIG. 11D is a graph showing the fold change of FL activity correlated with the fold change of the c-Myc phosphorylation level (R2=0.93).

FIGS. 12A-12D illustrate the phosphorylation sensor detected the inhibitory effect of AT in intact SKBR3 cells.

FIG. 12A is a digital image showing SK-BR-3 cells stably expressing the c-Myc phosphorylation sensor (SK ST) or the full-length FL (SK FST) treated with AT at 0 μM, 3 μM, 5 μM, 20 μM, and 50 μM for 18 h. Cells were imaged with D-Luc in the IVIS 50 BLI system.

FIG. 12B is a graph showing the FL activities of sensor ST and FL ST cells normalized to the total protein content measured from each cell lysate. The normalized FL activities of cells treated with AT and cells treated without AT were compared to obtain the fold change.

FIG. 12C is a digital image of a Western blot analysis of the endogenous level of phospho T58/S62 of c-Myc, c-Myc protein, and β-actin on AT treatment.

FIG. 12D is a graph showing the fold change of FL activity was correlated with the fold change of the c-Myc phosphorylation level (R2=0.88).

FIGS. 13A-13D illustrate the phosphorylation sensor of the disclosure detected the inhibitory effect of AT in intact 293T cells.

FIG. 13A is a digital image showing 293T cells stably expressing the c-Myc phosphorylation sensor (293T ST) or the full-length FL (293T FST) treated with AT at 0 μM, 3 μM, 5 μM, 10 μM, 20 μM, and 40 μM for 18 h. Cells were imaged with D-Luc in the IVIS 50 BLI system.

FIG. 13B is a graph showing the FL activities of sensor ST and FL ST cells normalized to the total protein content measured from each cell lysate. The normalized FL activities of cells treated with AT and cells treated without AT were compared to obtain the fold change.

FIG. 13C is a digital image showing a Western blot analysis of the endogenous level of phospho T58/S62 of c-Myc, c-Myc protein, and β-actin on AT treatment.

FIG. 13D is a graph showing the fold change of FL activity was correlated with the fold change of the c-Myc phosphorylation level (R2=0.87).

FIGS. 14A and 14B illustrate the S62A mutant sensor does not respond to phosphorylation inhibition of c-Myc.

FIG. 14A is a digital image showing the NFL-c-Myc WT construct or the NFL-c-MycS62A mutant construct transiently transfected into SKBR3 cells with GSK 35-433-CFL construct and subjected to PD98059 of indicated concentration for 1 h, followed by BLI.

FIG. 14B is a graph showing FL activity normalized to co-transfected RL activity and the total protein content and plotted against the drug concentration.

FIG. 15 is a graph showing a replotting of FIG. 8B as the photon output of the SK ST cells divided by that of the SK FST cells in the PBS or AT treatment group against days after treatment.

FIG. 16 illustrates the formula for nitazoxanide.

FIGS. 17A-17C illustrate in vitro validation of the assay system of the disclosure for the measurement of the decrease in luciferase activity when cells are exposed to nitazoxanide.

FIGS. 18A and 18B illustrates in vitro validation of the assay system of the disclosure for the measurement of the decrease in luciferase activity when cells are exposed to nitazoxanide.

FIG. 19 is a digital image of the results of a Western analysis showing that endogenous c-Myc levels are consistent with the detectable luciferase signal generated by the system of the disclosure.

FIG. 20 illustrates the measurement of cell viability and the toxicity of nitazoxanide.

FIG. 21 illustrates the imaging of implanted SK-ST and SK-FST in vivo and the effects of nitazoxanide treatment versus PBS control.

FIG. 22 schematically illustrates the constructs NFL-Myc and GSK3β-CFL, and the expression vector showing the sites of insertion of the two constructs therein.

FIG. 23 is a graph illustrating a summary of HTS results. The total number of compounds is plotted against the total potential hits that have a minimum of 30% inhibitory effect at 10 μM, and the total clean hits, which are potential hits but not cytotoxic or luciferase inhibitors. Contribution of each library to the total number of the three categories is also shown.

FIG. 24 illustrates the chemical structures of three compounds having an inhibitory effect on c-Myc. The percentage inhibition at 10 μM and the IC50 for each drug are also shown.

FIGS. 25A-25C illustrate the efficacy of NTZ in Cell culture. FIG. 25A: SK-ST and SK-FST stable cells were treated with NTZ at indicated concentrations and subjected to BLI. FIG. 25B: Plot of the fold change of FL activity after normalization to cell numbers at different concentrations of NTZ in SK-ST and SK-FST cells. FIG. 25C: SK-ST and SK-FST cells were treated with indicated drugs at different concentrations. FL activity of the SK-ST cells was normalized to that of the SK-FST cells and the fold-change of the normalized FL activity was plotted against the drug concentration.

FIGS. 26A and 26B illustrate the inhibition of endogenous c-Myc. FIG. 26A: Western blot analysis of SKBR3 cells after NTZ treatment was performed using phospho c-Myc, c-Myc protein, and β-actin antibodies. FIG. 26B: NTZ induced dose dependent inhibition of phospho c-Myc level in all three tumor cell lines, SKBR3 (IC₅₀=122 nM), lymphoma (IC₅₀=440 nM) and sarcoma cells (IC₅₀=12 nM). The fold-change of phospho c-Myc level was plotted against the log scale of NTZ concentration.

FIGS. 27A and 27B illustrate the efficacy of NTZ in a SK-ST subcutaneous tumor xenograft mouse model. FIG. 27A: SK-ST stable cells were subcutaneously implanted as indicated and treated with NTZ or CMC. Representative images of FL imaging at indicated days of treatment are shown. FIG. 27B: Photon output of SK-ST cells with NTZ or CMC treatment is plotted against days after implantation. * P<0.05.

FIGS. 28A and 28B illustrate NTZ inhibition of tumor growth. FIG. 28A: Representative photographs of removed tumors from CMC or NTZ treated mice 27 days after implantation. FIG. 28B: Plot of caliper measured tumor sizes of NTZ or CMC treated groups at different days after implantation. *P<0.05, **P<0.01.

FIGS. 29A and 29B illustrate immunohistochemistry and western blot analysis of NTZ treated tumor samples. FIG. 29A: Representative images of H&E, Ki67 and caspase-3 IHC staining of tumor tissue samples from each group. FIG. 29B: Western blot analysis of tumor tissue samples using phospho c-Myc, c-Myc and α-tubulin antibodies.

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. Patent law and can mean “ includes,” “including,” and the like; “consisting essentially of or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

### Abbreviations

FL, Firefly luciferase; hRL, human codon-optimized split Renilla luciferase; AT, Atorvastatin; Clz, coelenterazine; D-luc, D-luciferin; BLI, bioluminescence imaging; NTZ, nitazoxanide.

### Definitions

In describing and claiming the disclosed subject matter, the following terminology will be used in accordance with the definitions set forth below.

The term “generating an image” as used herein refers to acquiring a detectable signal generated from a luciferase light source according to the present disclosure and determining the location of the source in a cell or an animal or human tissue.

The term “c-Myc” as used herein refers to a transcription factor associated with a number of different cancers. It is a 439 amino acid, 64 kDa proteins, with O-linked glycosylation and phosphorylation sites characterized by N-terminal domains, termed Myc boxes, which are found in the closely related protein N-Myc and L-Myc. The C-terminal region contains a dimerization motif, termed helix-loop-helix leucine zipper, which permits homotypic or heterotypic dimerization. c-Myc is known to interact with a host of other cellular proteins, including MM-1, v-raf, c-Raf, SMAD2, MEK1, ReIA, .alpha.-tubulin, TRRAP, Smad3, p73, Tata box binding protein, Transcription factor IIF (α-subunit), p107, Nuclear transcription factor Y γ-subunit, Max-like protein X, CBF-C/NF-YB, BRAC1, YY1, Zinc finger protein 151, N-Myc interactor, Pam, Transcription factor AP-2β, Retinoblastoma 1, JNK1, SMARCB1, p34cdc2, AMY-1, HSP 90A, Tubulin α-2, Tubulin a (ubiquitous), Zinc finger protein 151, Tubulin α-8, ERK5, Tubulin α (brain specific), Nuclear transcription factor Y β-subunit, Tubulin α-1, and Max interacting protein 1.

Expression of c-Myc is tightly regulated by external signals. The resting cell expresses little c-Myc, whereas cells stimulated by growth factors dramatically increase c-Myc expression. Abnormal expression of c-Myc invokes p19Arf- and p53-dependent pathways, which eliminate such cells by induction of apoptosis. c-Myc is required for normal embryonic development. However, activation of the c-myc gene in adult cells can lead to development of cancers. Chromosomal translocations, as in the case of Burkitt's lymphoma, activate transcription of the c-myc gene by relocating it in the proximity of highly transcribed immunoglobulin genes. c-myc gene amplification (50-200 copies) also is found in cancer cells. Other mechanisms of c-Myc over-expression include increased transcription, removal of 3′-UTR destabilizing sequences, retroviral insertion, and post-translational modification.

The term “GSK3β” as used herein refers to a protein originally identified by its phosphorylation of glycogen synthase as described in Woodgett et al., (1991) Trends Biochem. Sci. 16: 177-181. Synonyms of GSK3 are tau protein kinase I (TPK I), FA kinase, and kinase F A. Mammalian forms of GSK3 have been cloned as described in Woodgett (1990) EMBO J. 9: 2431-2438 (1990). Truncated polypeptides of the disclosure possess one or more of the bioactivities of the GSK3 protein, including kinase activities such as polymerizing tau protein, or phosphorylating glycogen synthase, for example. Thus, truncated GSK3 polypeptides useful in the sensors of the disclosure can have sequence identity of at least 40%, preferably 50%, preferably 60%, preferably 70%, more preferably 80%, and most preferably 90% to the amino acid sequence of the native protein, wherever derived, from human or non-human sources. The polynucleotides encoding a GSK3 polypeptide can have 60%, preferably 70%, more preferably 80%, more preferably 90% and most preferably 95% sequence identity to a native polynucleotide sequence of GSK3. Also included, therefore, are alleles and variants of the native polynucleotide sequence such that the polynucleotide encodes an amino acid sequence with substitutions, deletions, or insertions, as compared to the native sequence.

The term “complemented FL activity” as used herein refers to the luciferase activity generated by the association of two polypeptides derived from a luciferase that, when in proximity to one another cooperatively interact to provide a detectable luciferase-generated signal. While the term “FL” refers to Firefly luciferase, it is contemplated to be within the scope of the disclosure for the luminescent activity to be provided by the complementation and cooperative interaction of two polypeptides from a luciferase other than derived from the firefly. It is further contemplated that the interacting polypeptides may be derived from luciferases from two different species. The term may also apply to other species of luciferase or variant thereof known in the art including, but not limited to, a human codon-optimized Renilla luciferase.

The terms “polypeptide” and “protein” as used herein refer to a polymer of amino acids of three or more amino acids in a serial array, linked through peptide bonds. The term “polypeptide” includes proteins, protein fragments, protein analogues, oligopeptides and the like. The term “polypeptides” contemplates polypeptides as defined above that are encoded by nucleic acids, produced through recombinant technology, isolated from an appropriate source such as a bird, or are synthesized. The term “polypeptides” further contemplates polypeptides as defined above that include chemically modified amino acids or amino acids covalently or non-covalently linked to labeling ligands.

The term “fragment” as used herein to refer to a nucleic acid (e.g., cDNA) refers to an isolated portion of the subject nucleic acid constructed artificially (e.g., by chemical synthesis) or by cleaving a natural product into multiple pieces, using restriction endonucleases or mechanical shearing, or a portion of a nucleic acid synthesized by PCR, DNA polymerase or any other polymerizing technique well known in the art, or expressed in a host cell by recombinant nucleic acid technology well known to one of skill in the art. The term “fragment” as used herein may also refer to an isolated portion of a polypeptide, wherein the portion of the polypeptide is cleaved from a naturally occurring polypeptide by proteolytic cleavage by at least one protease, or is a portion of the naturally occurring polypeptide synthesized by chemical methods well known to one of skill in the art or expressed from a recombinant nucleic acid.

The term “expressed” or “expression” as used herein refers to the transcription from a gene to give an RNA nucleic acid molecule at least complementary in part to a region of one of the two nucleic acid strands of the gene. The term “expressed” or “expression” as used herein also refers to the translation from said RNA nucleic acid molecule to give a protein or polypeptide or a portion thereof.

The term “coding region” as used herein refers to a continuous linear arrangement of nucleotides that may be translated into a protein. A full-length coding region is translated into a full length protein; that is, a complete protein as would be translated in its natural state absent any post-translational modifications. A full length coding region may also include any leader protein sequence or any other region of the protein that may be excised naturally from the translated protein.

The terms “percent sequence identity” or “percent sequence similarity” as used herein refer to the degree of sequence identity between two nucleic acid sequences or two amino acid sequences as determined using the algorithm of Karlin & Attschul (1990) Proc. Natl. Acad. Sci. 87: 2264-2268, modified as in Karlin & Attschul (1993) Proc. Natl. Acad. Sci. 90: 5873-5877. Such an algorithm is incorporated into the NBLAST and XBLAST programs of Attschul et al. (1990) T. Mol. Biol. Q15: 403-410. BLAST nucleotide searches are performed with the NBLAST program, score=100, word length=12, to obtain nucleotide sequences homologous to a nucleic acid molecule of the invention. BLAST protein searches are performed with the XBLAST program, score=50, word length=3, to obtain amino acid sequences homologous to a reference polypeptide. To obtain gapped alignments for comparison purposes, Gapped BLAST is utilized as described in Attschul et al. (1997) Nuc. Acids Res. 25: 3389-3402. When utilizing BLAST and Gapped BLAST programs, the default parameters of the respective programs (e.g., XBLAST and NBLAST) are used.

The term “nucleic acid vector” as used herein refers to a natural or synthetic single or double stranded plasmid or viral nucleic acid molecule that can be transfected or transformed into cells and replicate independently of, or within, the host cell genome. A circular double stranded plasmid can be linearized by treatment with an appropriate restriction enzyme based on the nucleotide sequence of the plasmid vector. A nucleic acid can be inserted into a vector by cutting the vector with restriction enzymes and ligating the pieces together. The nucleic acid molecule can be RNA or DNA.

The term “vector” as used herein refers to a polynucleotide comprised of single strand, double strand, circular, or supercoiled DNA or RNA. A typical vector may be comprised of the following elements operatively linked at appropriate distances for allowing functional gene expression: replication origin, promoter, enhancer, 5′ mRNA leader sequence, ribosomal binding site, nucleic acid cassette, termination and polyadenylation sites, and selectable marker sequences. One or more of these elements may be omitted in specific applications. The nucleic acid cassette can include a restriction site for insertion of the nucleic acid sequence to be expressed. In a functional vector the nucleic acid cassette contains the nucleic acid sequence to be expressed including translation initiation and termination sites.

A vector is constructed so that the particular coding sequence is located in the vector with the appropriate regulatory sequences, the positioning and orientation of the coding sequence with respect to the control sequences being such that the coding sequence is transcribed under the “control” of the control or regulatory sequences. Modification of the sequences encoding the particular protein of interest may be desirable to achieve this end. For example, in some cases it may be necessary to modify the sequence so that it may be attached to the control sequences with the appropriate orientation; or to maintain the reading frame. The control sequences and other regulatory sequences may be ligated to the coding sequence prior to insertion into a vector. Alternatively, the coding sequence can be cloned directly into an expression vector which already contains the control sequences and an appropriate restriction site which is in reading frame with and under regulatory control of the control sequences.

The term “expression vector” as used herein refers to a nucleic acid vector that may further include at least one regulatory sequence operably linked to a nucleotide sequence coding for a polypeptide or a protein protein. Regulatory sequences are well recognized in the art and may be selected to ensure good expression of the linked nucleotide sequence without undue experimentation by those skilled in the art. As used herein, the term “regulatory sequences” includes promoters, enhancers, and other elements that may control expression. Standard molecular biology textbooks such as Sambrook et al. Eds “Molecular Cloning: A Laboratory Manual” 2nd ed. Cold Spring Harbor Press (1989) may be consulted to design suitable expression vectors, promoters, and other expression control elements. It should be recognized, however, that the choice of a suitable expression vector depends upon multiple factors including the choice of the host cell to be transformed and/or the type of protein to be expressed.

The term “expression cassette” as used herein refers to one or more nucleotide sequences operably linked to a promoter region that can be transcribed to a provide a single mRNA transcript that may then be translated to at least one polypeptide.

The terms “transformation” and “transfection” as used herein refer to the process of inserting a nucleic acid into a host. Many techniques are well known to those skilled in the art to facilitate transformation or transfection of a nucleic acid into a prokaryotic or eukaryotic organism. These methods involve a variety of techniques, such as treating the cells with high concentrations of salt such as, but not only, a calcium or magnesium salt, an electric field, detergent, or liposome mediated transfection, to render the host cell competent for the uptake of the nucleic acid molecules.

The term “genetically modified cell” refers to a cell that has a new combination of nucleic acid segments that are not covalently linked to each other in nature. A new combination of nucleic acid segments can be introduced into an organism using a wide array of nucleic acid manipulation techniques available to those skilled in the art. A recombinant cell can be a single eukaryotic cell, or a single prokaryotic cell, or a mammalian cell. The recombinant cell can harbor a vector that is extragenomic. An extragenomic nucleic acid vector does not insert into the cell's genome. A recombinant cell can further harbor a vector or a portion thereof that is intragenomic. The term intragenomic defines a nucleic acid construct incorporated within the recombinant cell's genome.

The term “small molecule” as used herein refers to compositions that have a molecular weight of less than about 5 kDa and most preferably less than about 4 kDa. Small molecules can be, e.g., nucleic acids, peptides, polypeptides, peptidomimetics, carbohydrates, lipids or other organic or inorganic molecules.

The term “ligand” as used herein refers to a molecule, a small molecule, a biomolecule, a drug, a peptide, a polypeptide, a protein, a protein complex, an antibody, a nucleic acid, or a cell.

The terms “operatively linked” or “operably linked” or “operatively joined” or the like, as used herein refer to chimeric proteins, polypeptide sequences that are placed in a physical and functional relationship to each other, chimeric oligonucleotides or polynucleotides that are placed in a physical and functional relationship to each other. The terms “operably” or “operatively linked” as used herein can further refer to the configuration of the coding and control sequences so as to perform the desired function. Thus, control sequences operably linked to a coding sequence are capable of effecting the expression of the coding sequence. A coding sequence is operably linked to or under the control of transcriptional regulatory regions in a cell when DNA polymerase will bind the promoter sequence and transcribe the coding sequence into mRNA that can be translated into the encoded protein. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

The term “conservatively modified variation,” as used herein in reference to a particular polynucleotide sequence, refers to different polynucleotide sequences that encode identical or essentially identical amino acid sequences, or where the polynucleotide does not encode an amino acid sequence, to essentially identical sequences. Because of the degeneracy of the genetic code, a large number of functionally identical polynucleotides encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleotide sequence variations are “silent variations,” which can be considered a species of “conservatively modified variations.” As such, it will be recognized that each polynucleotide sequence disclosed herein as encoding a fluorescent protein variant also describes every possible silent variation. It will also be recognized that each codon in a polynucleotide, except AUG, which is ordinarily the only codon for methionine, and UUG, which is ordinarily the only codon for tryptophan, can be modified to yield a functionally identical molecule by standard techniques. Accordingly, each silent variation of a polynucleotide that does not change the sequence of the encoded polypeptide is implicitly described herein. Furthermore, it will be recognized that individual substitutions, deletions or additions that alter, add or delete a single amino acid or a small percentage of amino acids (typically less than 5%, and generally less than 1%) in an encoded sequence can be considered conservatively modified variations, provided alteration results in the substitution of an amino acid with a chemically similar amino acid.

Conservative amino acid substitutions providing functionally similar amino acids are well known in the art, including the following six groups, each of which contains amino acids that are considered conservative substitutes for each another: 1) Alanine (Ala, A), Serine (Ser, S), Threo nine (Thr, T); 2) Aspartic acid (Asp, D), Glutamic acid (Glu, E); 3) Asparagine (Asn, N), Glutamine (Gln, Q); 4) Arginine (Arg, R), Lysine (Lys, K); 5) Isoleucine (Ile, I), Leucine (Leu, L), Methionine (Met, M), Valine (Val, V); and 6) Phenylalanine (Phe, F), Tyrosine (Tyr, Y), Tryptophan (Trp, V).

Two or more amino acid sequences or two or more nucleotide sequences are considered to be “substantially identical” or “substantially similar” if the amino acid sequences or the nucleotide sequences share at least 80% sequence identity with each other, or with a reference sequence over a given comparison window. Thus, substantially similar sequences include those having, for example, at least 85% sequence identity, at least 90% sequence identity, at least 95% sequence identity, or at least 99% sequence identity.

The term “cancer”, as used herein shall be given its ordinary meaning and is a general term for diseases in which abnormal cells divide without control. Cancer cells can invade nearby tissues and can spread through the bloodstream and lymphatic system to other parts of the body.

There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. Generally, a solid tumor is an abnormal mass of tissue that usually does not contain cysts or liquid areas (some brain tumors do have cysts and central necrotic areas filled with liquid). A single tumor may even have different populations of cells within it with differing processes that have gone awry. Solid tumors may be benign (not cancerous), or malignant (cancerous). Different types of solid tumors are named for the type of cells that form them. Examples of solid tumors are sarcomas, carcinomas, and lymphomas. Leukemias (cancers of the blood) generally do not form solid tumors.

Representative cancers include, but are not limited to, bladder cancer, breast cancer, colorectal cancer, endometrial cancer, head & neck cancer, leukemia, lung cancer, lymphoma, melanoma, non-small-cell lung cancer, ovarian cancer, prostate cancer, testicular cancer, uterine cancer, cervical cancer.

The term “DNA amplification” as used herein refers to any process that increases the number of copies of a specific DNA sequence by enzymatically amplifying the nucleic acid sequence. A variety of processes are known. One of the most commonly used is the polymerase chain reaction (PCR), which is defined and described in later sections below. The PCR process of Mullis is described in U.S. Pat. Nos. 4,683,195 and 4,683,202. PCR involves the use of a thermostable DNA polymerase, known sequences as primers, and heating cycles, which separate the replicating deoxyribonucleic acid (DNA), strands and exponentially amplify a gene of interest. Any type of PCR, such as quantitative PCR, RT-PCR, hot start PCR, LAPCR, multiplex PCR, touchdown PCR, etc., may be used. Advantageously, real-time PCR is used. In general, the PCR amplification process involves an enzymatic chain reaction for preparing exponential quantities of a specific nucleic acid sequence. It requires a small amount of a sequence to initiate the chain reaction and oligonucleotide primers that will hybridize to the sequence. In PCR the primers are annealed to denatured nucleic acid followed by extension with an inducing agent (enzyme) and nucleotides. This results in newly synthesized extension products. Since these newly synthesized sequences become templates for the primers, repeated cycles of denaturing, primer annealing, and extension results in exponential accumulation of the specific sequence being amplified. The extension product of the chain reaction will be a discrete nucleic acid duplex with a termini corresponding to the ends of the specific primers employed.

The term “fragment” of a molecule such as a protein or nucleic acid as used herein refers to any portion of the amino acid or nucleotide genetic sequence.

The term “polymerase chain reaction” or “PCR” as used herein refers to a thermocyclic, polymerase-mediated, DNA amplification reaction. A PCR typically includes template molecules, oligonucleotide primers complementary to each strand of the template molecules, a thermostable DNA polymerase, and deoxyribonucleotides, and involves three distinct processes that are multiply repeated to effect the amplification of the original nucleic acid. The three processes (denaturation, hybridization, and primer extension) are often performed at distinct temperatures, and in distinct temporal steps. In many embodiments, however, the hybridization and primer extension processes can be performed concurrently. The nucleotide sample to be analyzed may be PCR amplification products provided using the rapid cycling techniques described in U.S. Pat. Nos. 6,569,672; 6,569,627; 6,562,298; 6,556,940; 6,569,672; 6,569,627; 6,562,298; 6,556,940; 6,489,112; 6,482,615; 6,472,156; 6,413,766; 6,387,621; 6,300,124; 6,270,723; 6,245,514; 6,232,079; 6,228,634; 6,218,193; 6,210,882; 6,197,520; 6,174,670; 6,132,996; 6,126,899; 6,124,138; 6,074,868; 6,036,923; 5,985,651; 5,958,763; 5,942,432; 5,935,522; 5,897,842; 5,882,918; 5,840,573; 5,795,784; 5,795,547; 5,785,926; 5,783,439; 5,736,106; 5,720,923; 5,720,406; 5,675,700; 5,616,301; 5,576,218 and 5,455,175, the disclosures of which are incorporated by reference in their entireties. Other methods of amplification include, without limitation, NASBR, SDA, 3SR, TSA and rolling circle replication. It is understood that, in any method for producing a polynucleotide containing given modified nucleotides, one or several polymerases or amplification methods may be used. The selection of optimal polymerization conditions depends on the application.

The terms “therapeutic agent” or “chemotherapeutic agent” as used herein refers to a compound or a derivative thereof that can interact with a cancer cell, thereby reducing the proliferative status of the cell and/or killing the cell.

The terms “therapeutically effective amount” and “therapeutic dose” as used herein refer to that amount of the compound being administered which will relieve to some extent one or more of the symptoms of a disease, a condition, or a disorder being treated. In reference to cancer or pathologies related to unregulated cell division, a therapeutically effective amount refers to that amount which has the effect of (1) reducing the size of a tumor, (2) inhibiting (that is, slowing to some extent, preferably stopping) aberrant cell division, for example cancer cell division, (3) preventing or reducing the metastasis of cancer cells, and/or, (4) relieving to some extent (or, preferably, eliminating) one or more symptoms associated with a pathology related to or caused in part by unregulated or aberrant cellular division, including for example, cancer, or angiogenesis.

As used herein, the terms “subject” and “patient” includes humans, mammals (e.g., cats, dogs, horses, etc.), living cells, and other living organisms. A living organism can be as simple as, for example, a single eukaryotic cell or as complex as a mammal. Typical hosts to which embodiments of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. In some embodiments, a system includes a sample and a subject. The term “living host” refers to host or organisms noted above that are alive and are not dead. The term “living host” refers to the entire host or organism and not just a part excised (e.g., a liver or other organ) from the living host.

The term “promoter” as used herein refers to the DNA sequence that determines the site of transcription initiation from an RNA polymerase. A “promoter-proximal element” may be a regulatory sequence within about 200 base pairs of the transcription start site.

The term “recombinant cell” refers to a cell that has a new combination of nucleic acid segments that are not covalently linked to each other in nature. A new combination of nucleic acid segments can be introduced into an organism using a wide array of nucleic acid manipulation techniques available to those skilled in the art. A recombinant cell can be a single eukaryotic cell, or a single prokaryotic cell, or a mammalian cell. The recombinant cell may harbor a vector that is extragenomic. An extragenomic nucleic acid vector does not insert into the cell's genome. A recombinant cell may further harbor a vector or a portion thereof that is intragenomic. The term intragenomic defines a nucleic acid construct incorporated within the recombinant cell's genome.

The terms “recombinant nucleic acid” and “recombinant DNA” as used herein refer to combinations of at least two nucleic acid sequences that are not naturally found in a eukaryotic or prokaryotic cell. The nucleic acid sequences include, but are not limited to, nucleic acid vectors, gene expression regulatory elements, origins of replication, suitable gene sequences that when expressed confer antibiotic resistance, protein-encoding sequences, and the like. The term “recombinant polypeptide” is meant to include a polypeptide produced by recombinant DNA techniques such that it is distinct from a naturally occurring polypeptide either in its location, purity or structure. Generally, such a recombinant polypeptide will be present in a cell in an amount different from that normally observed in nature.

The terms “heterologous” and “exogenous” as they relate to nucleic acid sequences such as coding sequences and control sequences denote sequences that are not normally associated with a region of a recombinant construct or with a particular chromosomal locus, and/or are not normally associated with a particular cell. Thus, a “heterologous” region of a nucleic acid construct is an identifiable segment of nucleic acid within or attached to another nucleic acid molecule that is not found in association with the other molecule in nature. For example, a heterologous region of a construct tip could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a host cell transformed with a construct which is not normally present in the host cell would be considered heterologous for purposes of this invention.

In some embodiments the promoter will be modified by the addition or deletion of sequences, or replaced with alternative sequences, including natural and synthetic sequences as well as sequences which may be a combination of synthetic and natural sequences. Many eukaryotic promoters contain two types of recognition sequences: the TATA box and the upstream promoter elements. The former, located upstream of the transcription initiation site, is involved in directing RNA polymerase to initiate transcription at the correct site, while the latter appears to determine the rate of transcription and is upstream of the TATA box. Enhancer elements can also stimulate transcription from linked promoters, but many function exclusively in a particular cell type. Many enhancer/promoter elements derived from viruses, e.g. the SV40, the Rous sarcoma virus (RSV), and CMV promoters are active in a wide array of cell types, and are termed “constitutive” or “ubiquitous.” The nucleic acid sequence inserted in the cloning site may have any open reading frame encoding a polypeptide of interest, with the proviso that where the coding sequence encodes a polypeptide of interest, it should lack cryptic splice sites which can block production of appropriate mRNA molecules and/or produce aberrantly spliced or abnormal mRNA molecules.

The termination region which is employed primarily will be one of convenience, since termination regions appear to be relatively interchangeable. The termination region may be native to the intended nucleic acid sequence of interest, or may be derived from another source.

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein.

Further definitions are provided in context below. Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein.

### Description

Multimodality molecular imaging has emerged as a key spectrum of technologies to advance our understanding of disease mechanisms and accelerate drug discovery and development (Willmann et al., (2008) Nat. Rev. Drug Discov. 7: 591-607). In particular, reporter gene imaging strategies based on protein-assisted complementation of split luciferases are emerging as powerful tools for detecting and quantifying induced protein interactions and functional protein modifications in vivo, such as ubiquitination and phosphorylation (Kaihara et al., (2003) Anal. Chem. 75: 4176-4181; Luker et al., (2003) Nat. Med. 9: 969-973; Chan et al. (2008) Cancer Res. 68: 216-226; Paulmurugan & Gambhir (2007) Anal. Chem. 79: 2346-2353). To non-invasively monitor and image phosphorylation-mediated c-Myc activation, a split Firefly luciferase (FL)-based sensor system is herein described in which the complementation of the split FL is induced by phosphorylation-mediated interaction between GSK3β and c-Myc. The complemented FL activity resulting from this interaction is specific to c-Myc phosphorylation and correlates with the steady-state and temporal regulation of c-Myc phosphorylation in cell culture. The sensor system also allows monitoring of c-Myc—targeted drug efficacy in intact cells and living mice. This new imaging sensor is suitable for investigating the role of functional c-Myc in cancer biology and can be useful in the discovery and development of new, more specific anti-c-Myc drugs.

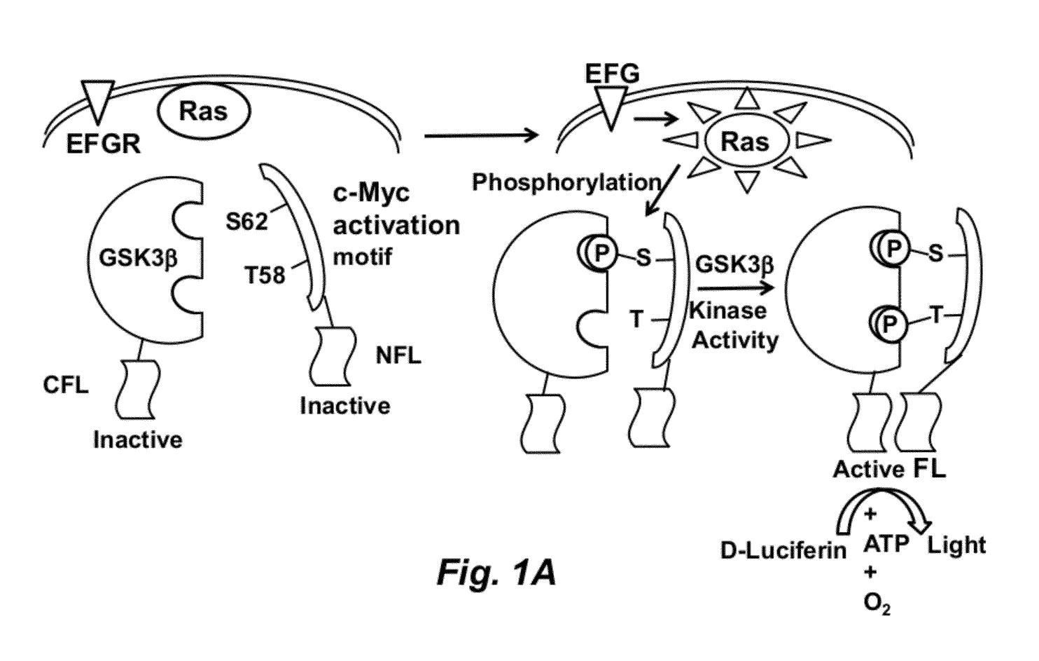

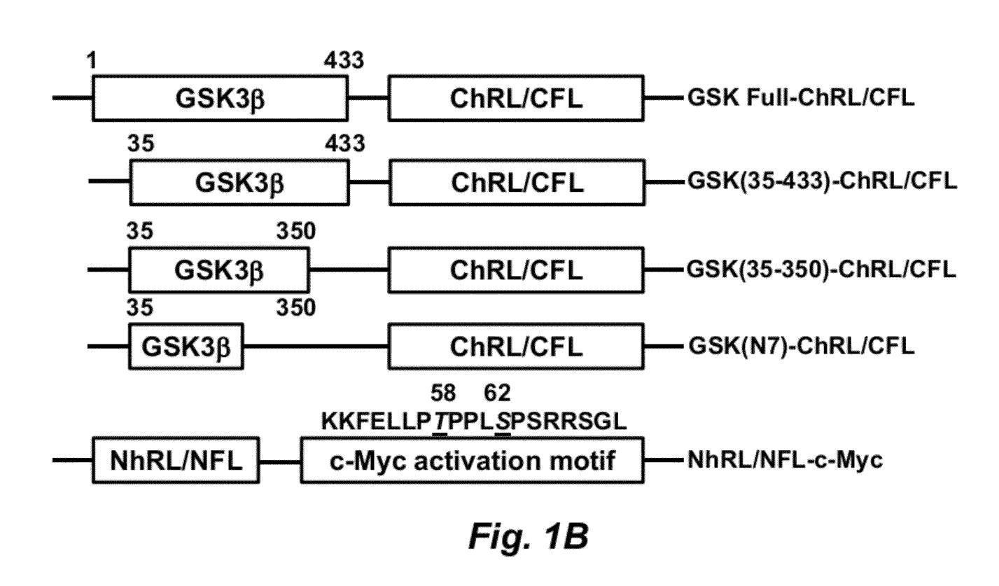

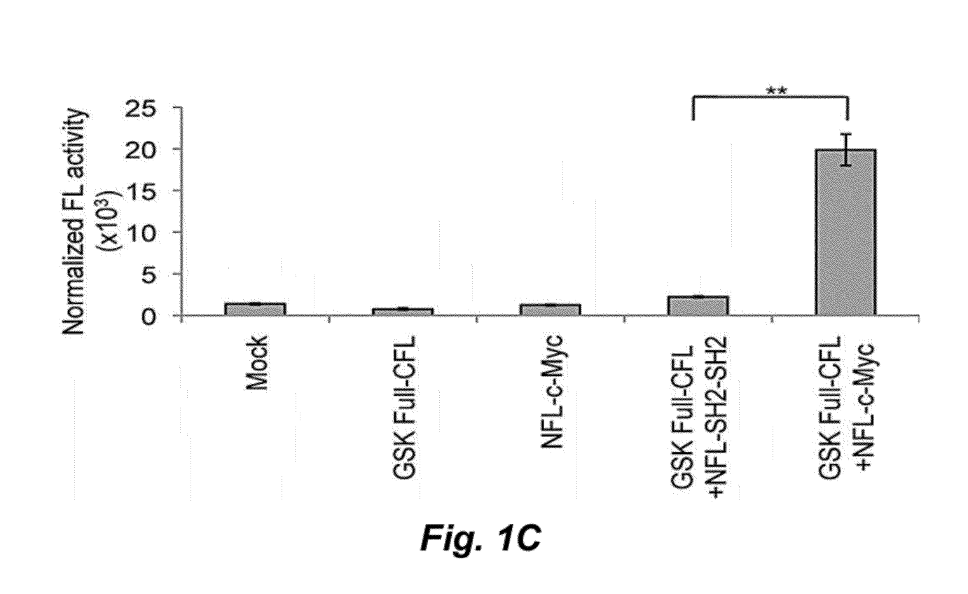

Because oncogenic activation of the c-Myc gene involves insertional mutagenesis, chromosomal translocation, and gene amplification (Vita & Henriksson (2006) Semin Cancer Biol 16(4):318-330), non-invasive monitoring of c-Myc activity at the transcriptional level using reporter genes is challenging. The phosphorylation-mediated activation of c-Myc protein contributes to the actual function of c-Myc in many normal and cancerous molecular processes. Thus, S62 phosphorylation is required for the interaction between GSK3β and c-Myc, making imaging the phospho signal and activation of c-Myc by detection of GSK3β-c-Myc interaction possible. Accordingly, the present disclosure encompasses an imaging sensor based on a protein-assisted split luciferase complementation strategy by fusing GSK3β fragments and a c-Myc activation motif with the C-terminal and N-terminal fragments of split FL, respectively. The resulting complemented FL activity is dependent on the interaction between the GSK3β fragment and the c-Myc activation motif (FIG. 1C).

The 35-433 aa fragment of GSK3β, but not other truncations, is sufficient to confer specificity to S62 phosphorylation of NFL-c-Myc. The deleted 1-34 aa fragment of GSK3β actually contains the autoinhibition site Ser-9, which prevents substrate binding to the phospho priming site (Dajani et al. (2001) Cell 105: 721-732); thus, the GSK 35-433-CFL fusion protein is not susceptible to the intrinsic inhibition of GSK3β in cancer cells.

Similarly, the 19 amino acid activation motif contains the phospho sites for c-Myc activation, but is not sufficient for ubiquitin-mediated proteasomal degradation (Muller & Eilers (2008) Ernst Schering Found Symp. Proc. 1: 99-113). Split RL fusion and split FL fusion constructs were also compared. The latter resulted in a greater photon output, which also may have an advantage over the hRL fusion in living subjects due to a more red-shifted wavelength, allowing greater light penetration through the body. Accordingly, GSK 35-433-CFL/NFL-c-Myc comprises a sensor that generates complemented FL activity, measurable through an imaging signal, on S62 phosphorylation of c-Myc.

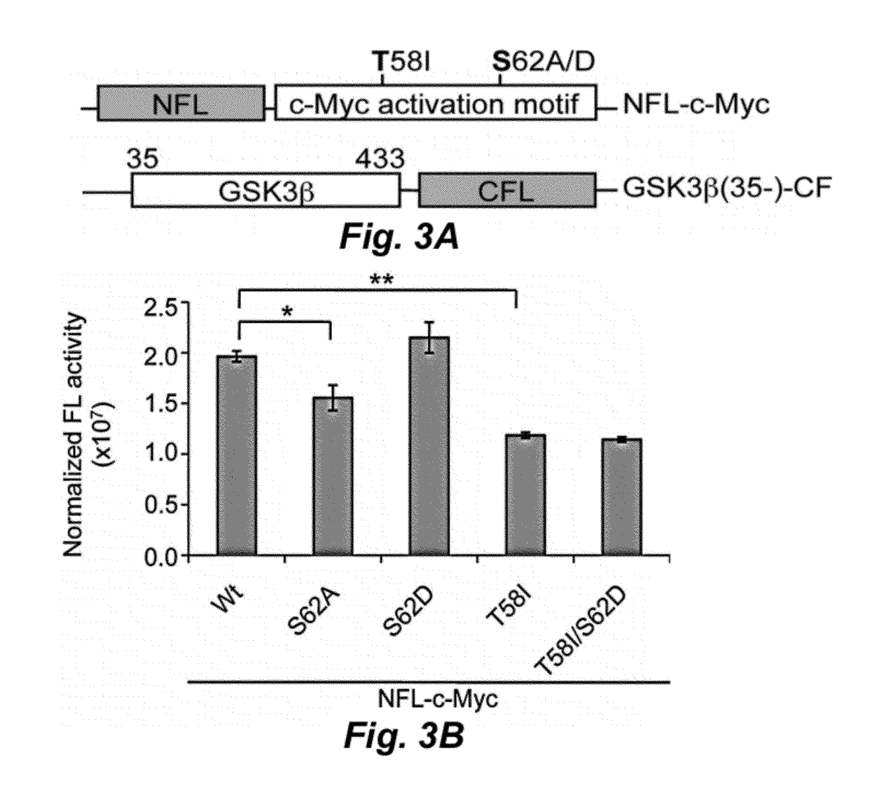

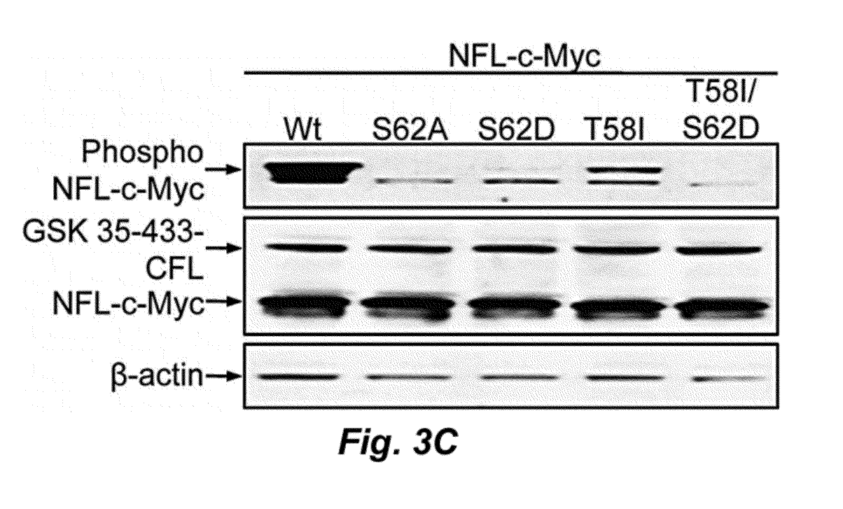

The phosphorylation dependence of the split FL complementation was further characterized by introducing a T581 mutation, and found that this resulted in a greater reduction of complemented FL activity than the S62A mutation, suggesting that T58 phosphorylation is more efficient in inducing reporter complementation. This also indicates that T58 phosphorylation is required for high-affinity binding of GSK3β to c-Myc, which in turn brings two inactive split FL fragments closer for complementation of FL reporter, as shown in FIG. 1A. However, the Western blot analysis indicated that T58 phosphorylation was dependent on S62 phosphorylation (FIG. 3C), which is consistent with the endogenous phosphorylation process of c-Myc on activation. Therefore, by including both phosphorylation sites within its constructs, the developed sensor enables imaging of S62 phosphorylation that induces efficient split FL complementation.

The sensor of the present disclosure has been validated in intact cells and in living mice, demonstrating that the sensor signal (i.e. complemented FL activity) is closely correlated with the endogenous c-Myc phosphorylation level in culture cells and living mice, either at steady-state, on serum stimulation, or on drug treatment. The results indicate that the sensor system provides a means to non-invasively image and quantify c-Myc phosphorylation and activation with high sensitivity in living subjects. Thus, this sensor can be useful in investigating the roles of phosphorylation in many c-Myc functions, including apoptosis, angiogenesis, and tumorigenesis (5), by non-invasively reporting the phosphorylation status in cells under varying levels of spatial and temporal regulation.

c-Myc is considered an attractive candidate for targeted cancer therapy because of its disregulation in most human cancers. The sensor system of the present disclosure may be used, therefore, to identify c-Myc—argeted molecular therapeutics by providing a rapid, noninvasive readout of c-Myc activity in various in vitro assays as well as high-throughput screening of chemical libraries, with subsequent further validation of their effects in xenograft or transgenic mouse models (the latter by exploiting the in vivo imaging capability of the sensor system of the present disclosure). Furthermore, the quantitative readout of c-Myc activity in living subjects on drug treatment can be useful for establishing future dosing parameters in preclinical models to guide initial clinical trials and eventual clinical applications. Sensor System for Imaging of c-Myc Phosphorylation.

The present disclosure encompasses a sensor system to image phosphorylation-induced interaction between GSK3β and c-Myc mediated by S62 phosphorylation of the c-Myc component. Protein-assisted split luciferase complementation systems were used for imaging protein—protein interactions in living subjects (Paulmurugan & Gambhir (2007) Anal. Chem. 79: 2346-2353; Paulmurugan et al., (2002) Proc. Natl. Acad. Sci. U.S.A. 99: 15608-15613; Paulmurugan & Gambhir (2003) Anal Chem 75: 1584-1589; Luker et al. (2004) Proc. Natl. Acad. Sci. U.S.A. 101: 12288-12293). Through fusions of an activation motif of c-Myc and a phosphor-recognition domain of GSK3[3 with split luciferase fragments (FIG. 1B), S62 phosphorylation results in the reporter fragment complementation by inducing the interaction between the c-Myc activation motif and the GSK3β recognition domain. Furthermore, T58 phosphorylation resulted in greater approximation of the split fragments and complementation of the reporter and restoration of bioluminescence, as schematically shown in FIG. 1A. Although it is contemplated that endogenous GSK3β and c-Myc proteins also may interact with the fused c-Myc activation motif and the GSK3β recognition domain, respectively; such competitive bindings do not result in reporter complementation.

The selected c-Myc activation motif of the sensor of the disclosure comprises the amino acids corresponding to positions 51-69 in the Myc homology box I (MBI) of c-Myc, which is part of the Myc transactivation domain that mediates c-Myc activation (Cowling & Cole (2006) Semin. Cancer Biol. 16: 242-252). This truncated region, however, is not sufficient for ubiquitin-mediated proteasomal degradation (Muller & Eilers (2008) Ernst Schering Found. Symp. Proc. 1:99-113).

Several truncations of GSK3[3 [GSK(N7), GSK(35-350), and GSK(35-433)] were constructed (FIG. 1B) based on the crystal structure and functional domains of GSK3β (Dajani et al. (2001) Cell 105: 721-732) for determination of the optimal sensitivity and specificity to c-Myc phosphorylation. Two types of split luciferase fragments: human codon-optimized split Renilla luciferase (hRL), and split FL, were used for fusion and compared for signal strengths (FIG. 1B). The split sites, NhRL229/ChRL230 and NFL398/CFL394, were chosen based on their optimal intrinsic complementation efficiencies.

To test the dependence of the split luciferase complementation on GSK3β-c-Myc interaction, the mock DNA (vector alone), a single sensor construct (GSK Full-CFL or NFL-c-Myc), a single sensor construct with a scrambled fusion construct (GSK Full-CFL/NFL-SH2-SH2), and paired sensor constructs (GSK Full-CFL/NFL-c-Myc) were introduced into SKBR3 breast cancer cells, as shown in FIG. 1C, in which c-Myc was constitutively activated (Robanus-Maandag et al. (2003) J. Pathol. 201: 75-82). No single sensor construct resulted in split FL complementation, whereas the pairing of GSK Full-CFL and NFL-c-Myc resulted in more than a 10-fold higher complemented FL activity compared with the pairing of GSK Full-CFL and NFL-SH2-SH2 (FIG. 1C).

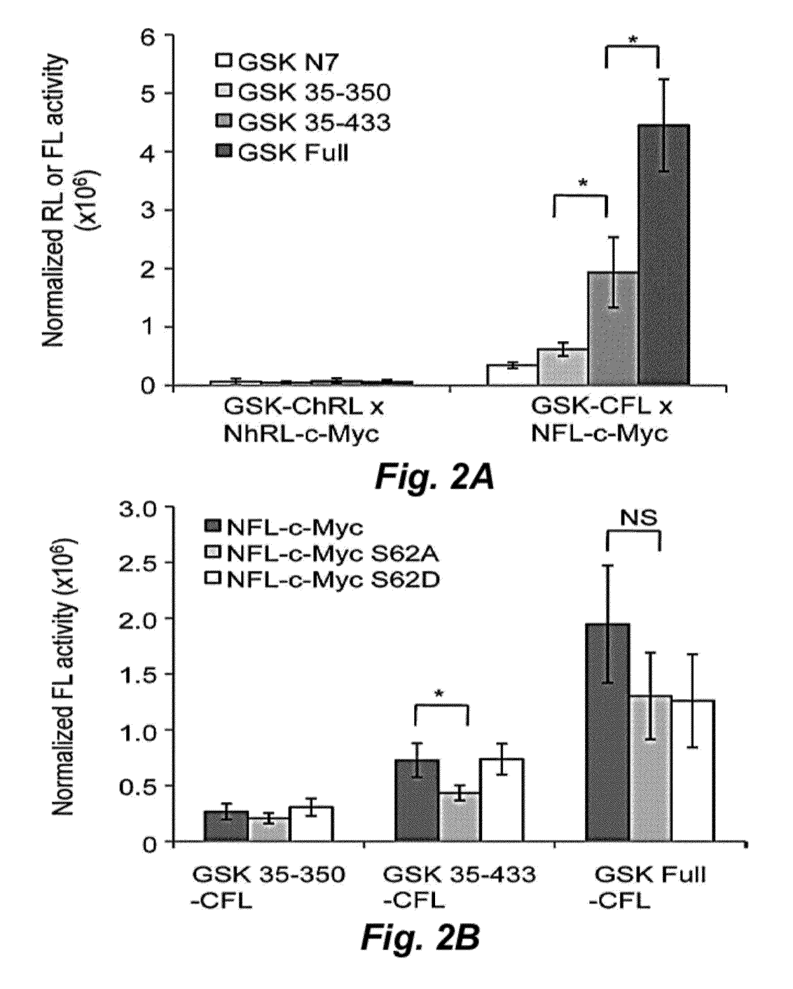

The signal strength of paired sensor constructs using split hRL fusion were also compared with those using split FL fusion. All paired split hRL fusion constructs resulted in much lower complemented luciferase activity than did the paired split FL fusion constructs (FIG. 2A), suggesting higher signal yield with the split FL fusion. Meanwhile, among the split FL fusion constructs, the pairing of different GSK3β truncations with NFL-c-Myc resulted in different levels of complemented FL activity, with the highest activity detected with the full-length GSK3β.

To test the specificity of different GSK3β truncations to c-Myc phosphorylation, the mutation S62A was introduced, which abolished phosphorylation, and the mutation S62D, which replicated the constitutive phosphorylation of NFL-c-Myc. Different CFL-fused GSK3β truncations were co-expressed with NFL-c-Myc, NFL-c-Myc S62A, or NFL-c-Myc S62D in SKBR3 cells, except GSK N7-CFL, which had very low signal yield (FIG. 2A). Compared with NFL-c-Myc, NFL-c-Myc S62A resulted in significant reduction of complemented FL activity when paired with GSK 35-433-CFL (P<0.05), but not when paired with GSK 35-350-CFL or GSK Full-CFL, and NFL-c-Myc S62D resulted in recovery of complemented FL activity when paired with GSK 35-433-CFL and GSK 35-350-CFL, but not when paired with GSK Full-CFL (FIG. 2B). These results showed that only GSK 35-433-CFL is able to confer specificity to S62 phosphorylation of NFL-c-Myc. Thus, the combination of GSK 35-433-CFL and NFL-c-Myc constitutes a particularly useful sensor compared to other combinations of constructs for detecting phosphorylation-mediated interaction between GSK3β and c-Myc.

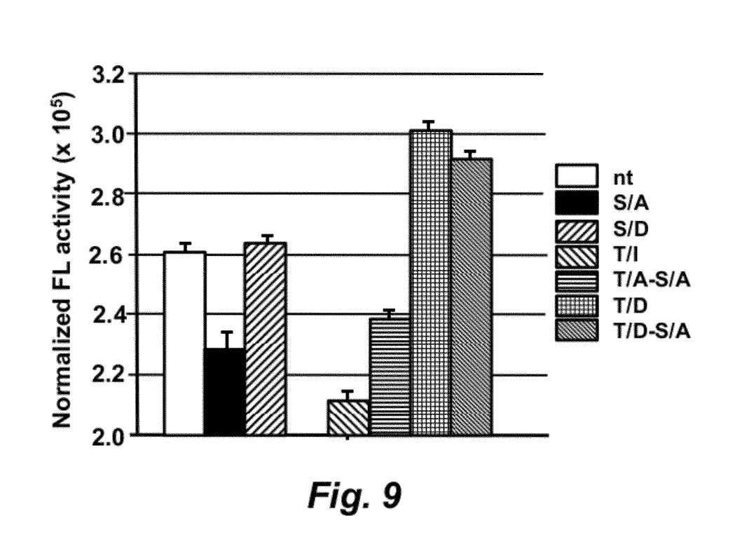

To further characterize the phosphorylation dependence of the complementation between GSK 35-433-CFL and NFL-c-Myc, mutation T581 was introduced (FIG. 3A), which frequently occurs in lymphomas to prevent c-Myc from T58 phosphorylation by GSK3β and subsequent degradation (Bhatia et al. (1993) Nat. Genet. 5: 56-61). T581 was also paired with the S62A or S62D mutation to compare the impact on reporter complementation of phosphorylation at T58 and that at S62 (FIG. 3B). Co-expression of GSK 35-433-CFL with the T581 mutation in SKBR3 cells resulted in a greater reduction in complemented FL activity than co-expression with the S62A mutation (FIG. 3B; P<0.01). The T581/S62A double mutations resulted in a similar reduction as the T581 single mutation (FIG. 9), whereas the T581/S62D double mutations did not result in recovery of the complemented FL activity, unlike the S62D single mutation (FIG. 3B).

These results indicated that depletion of T58 phosphorylation compromised S62 phosphorylation-induced NFL-c-Myc interaction with GSK 35-433-CFL and further complementation of the split FL. Western blot analysis of the cell lysates revealed the sensor phosphorylation status with a phospho c-Myc antibody against T58 and/or S62 (FIG. 3). The NFL-c-Myc was highly phosphorylated, whereas the S62A mutation abolished all phosphorylation, and the T581 mutation abolished it partially. The antibody did not recognize the phospho group of Asp, and thus the T58 phosphorylation in the S62D mutant was revealed as a faint band, and the T581/S62D mutant had no detectable band. Taken together, these results show that S62 phosphorylation of NFL-c-Myc is required for T58 phosphorylation, which in turn affects S62 phosphorylation-induced split FL complementation.

Steady-State Imaging of c-Myc Phosphorylation in Intact Cells.

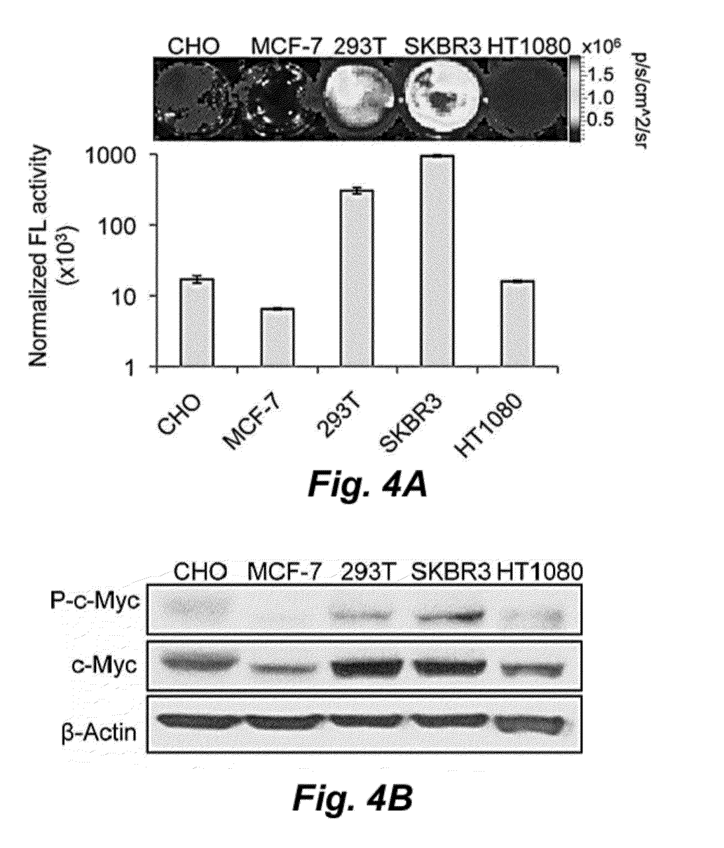

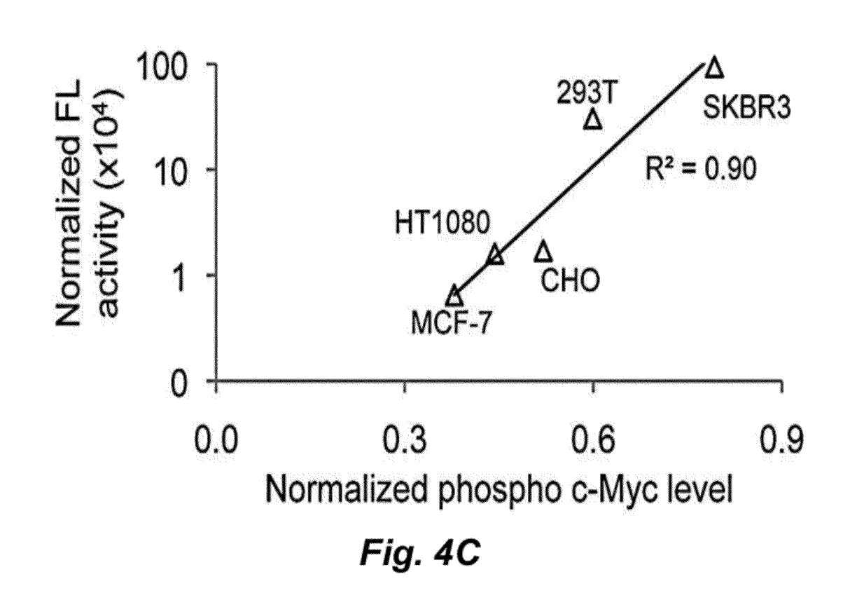

The sensor GSK 35-433-CFL/NFL-c-Myc was transiently transfected into Chinese hamster ovary (CHO), MCF-7, 293T, SKBR3, and HT1080 cells to test its ability in detecting the steady-state level of c-Myc phosphorylation. The complemented FL activity in CHO cells was 2.6-fold higher than that in MCF-7 cells, 17.9-fold lower than that in 293T cells, 54.9-fold lower than that in SKBR3 cells, and 1.1-fold higher than that in HT1080 cells (FIG. 4A). These sensor signals also were correlated with the steady-state differential levels of c-Myc phosphorylation in those cells (R₂=0.90; FIG. 4C), as revealed by Western blot analysis of the cell lysates (FIG. 4B).

Dynamic Imaging of c-Myc Activation on Serum Stimulation.

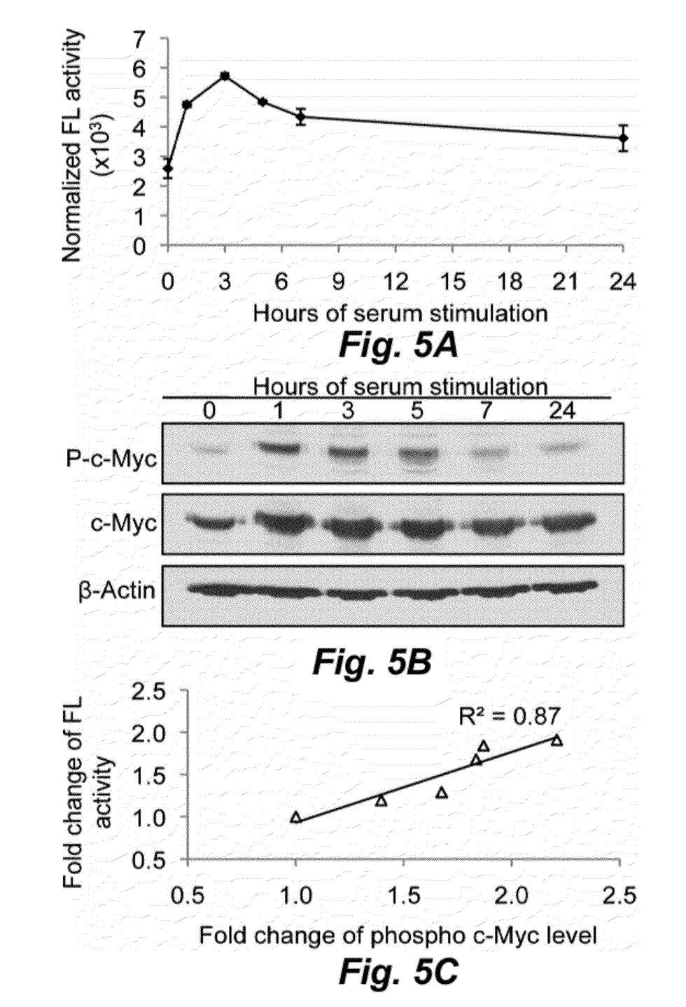

On growth factor stimulation, c-Myc exhibits temporal activation, followed by a rapid decline to basal levels (Yeh et al. (2004) Nat. Cell. Biol. 6:308-318). To test the sensor's ability to detect dynamic regulation of c-Myc phosphorylation, a growth stimulation assay was performed. CHO cells were transfected with the sensor and serum-starved for 24 h, and then stimulated with 20% serum for 0 h, 1 h, 3 h, and 5 h. The complemented FL activity increased after serum stimulation, peaked at 3 h, and then declined rapidly to the basal steady-state level by 24 h (FIG. 5A). This dynamic change in the sensor signal also was correlated with temporal regulation of the endogenous c-Myc phosphorylation level (R2=0.87; FIG. 5C), as revealed by Western blot analysis (FIG. 5B).

Imaging of c-Myc Phosphorylation Inhibition in Intact Cells.

To test the ability of the sensor to monitor c-Myc phosphorylation levels on drug treatment, the SKBR3 and 293T stable cells (SK ST and 293T ST) were established that constitutively express the sensor. SKBR3 and 293T stable cells were also constructed that constitutively express the full-length FL (SK FST and 293T FST) for the control of direct drug effect on FL activity. The SK ST cells were treated with the MAP kinase inhibitors (PD98059 and U0126) and Atorvastatin (AT), all of which induced dose-dependent reduction of complemented FL activity after normalization to their effect on the FL activity of SK FST cells.

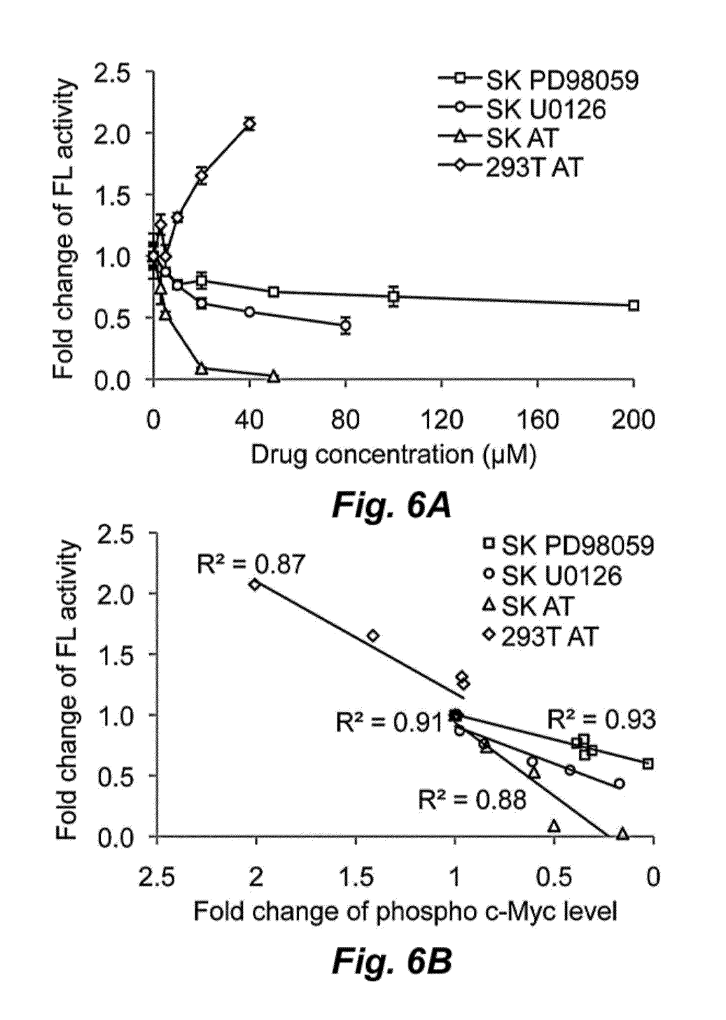

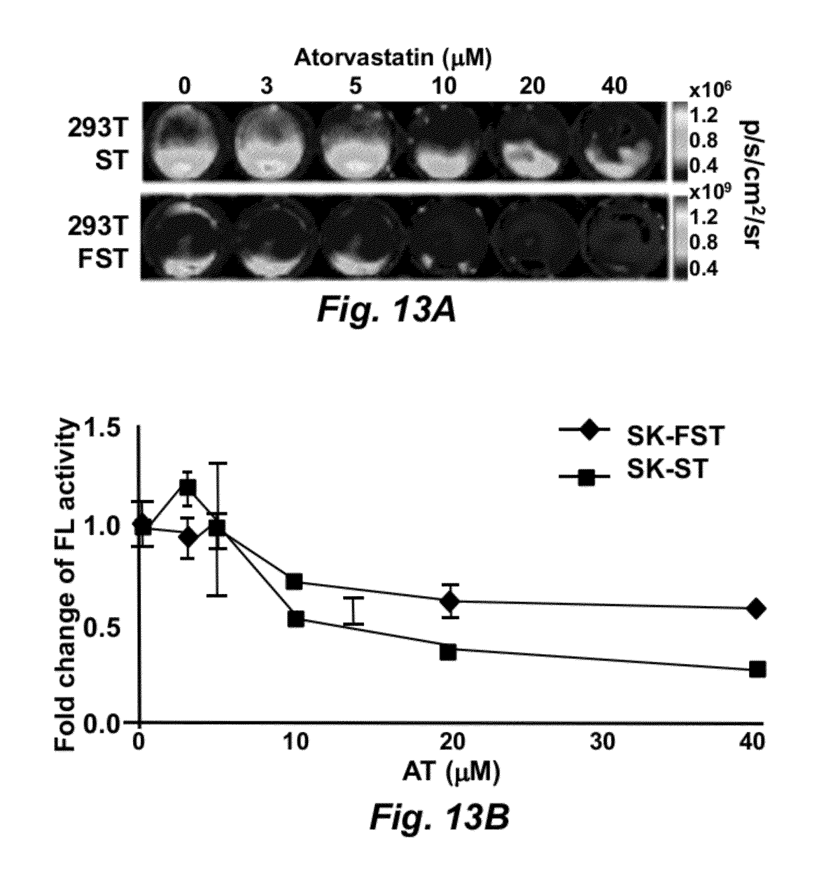

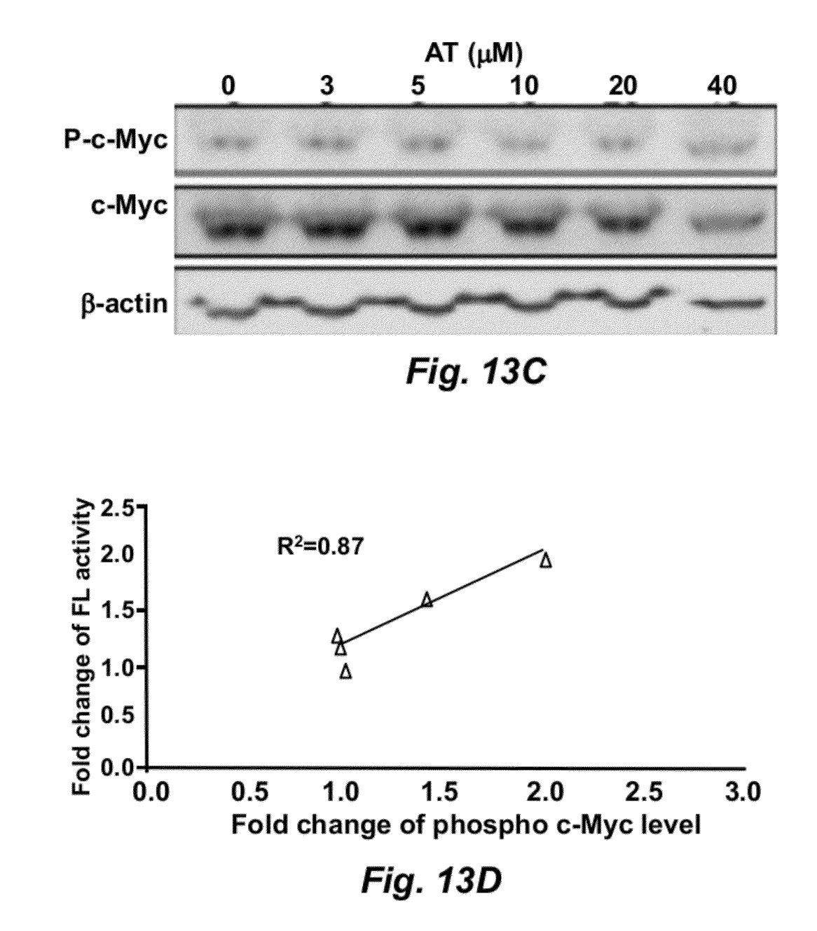

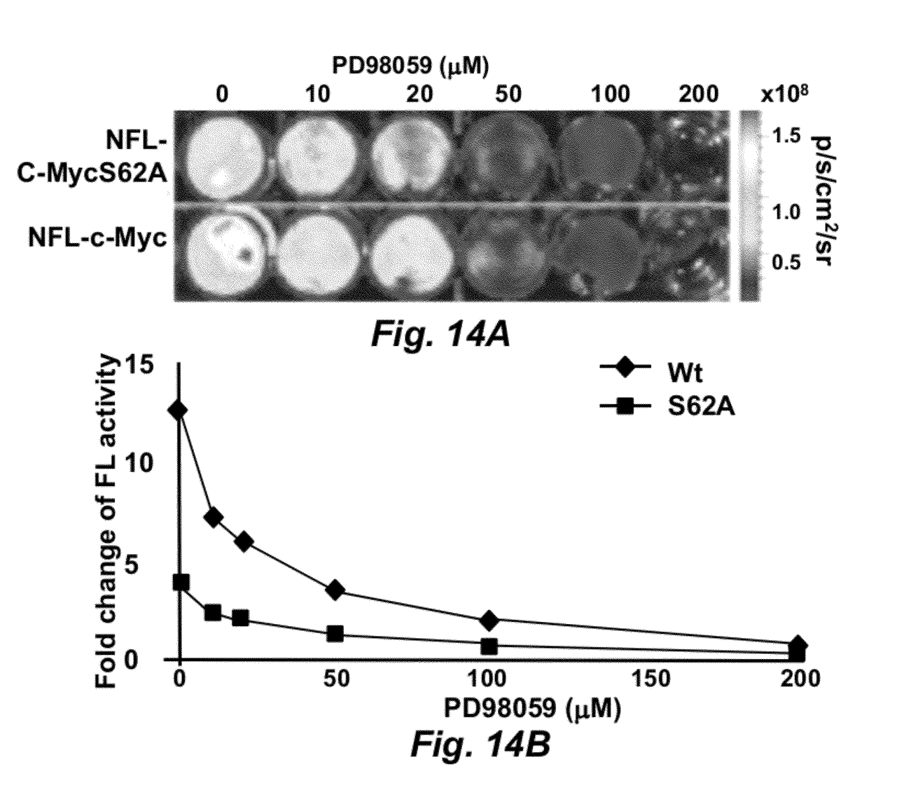

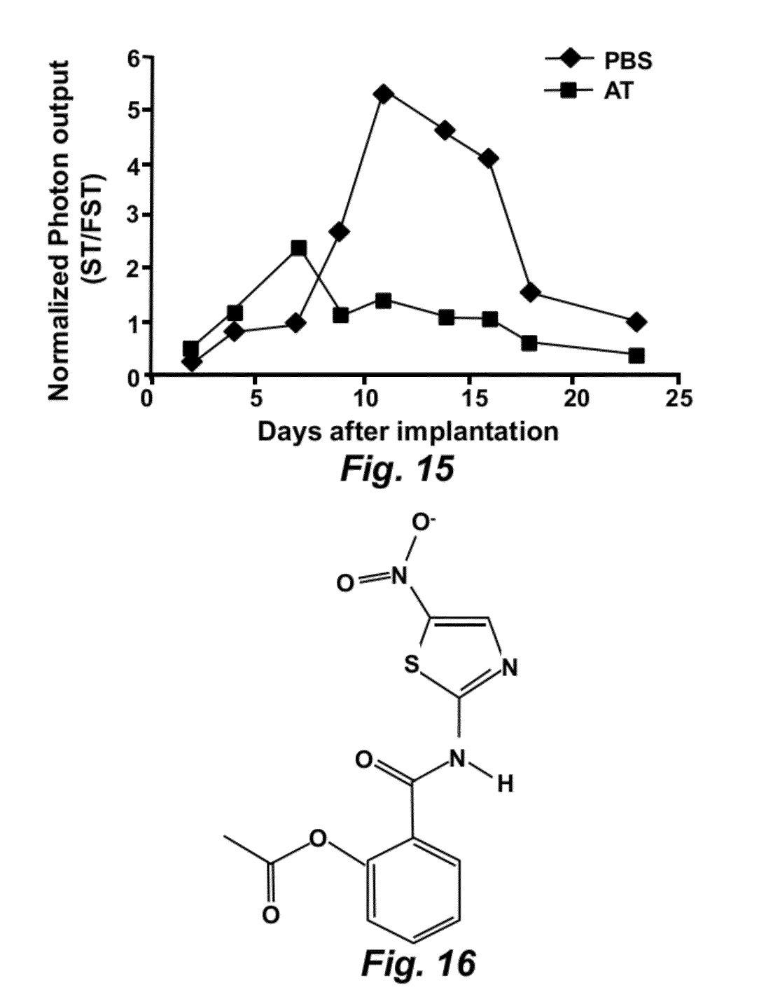

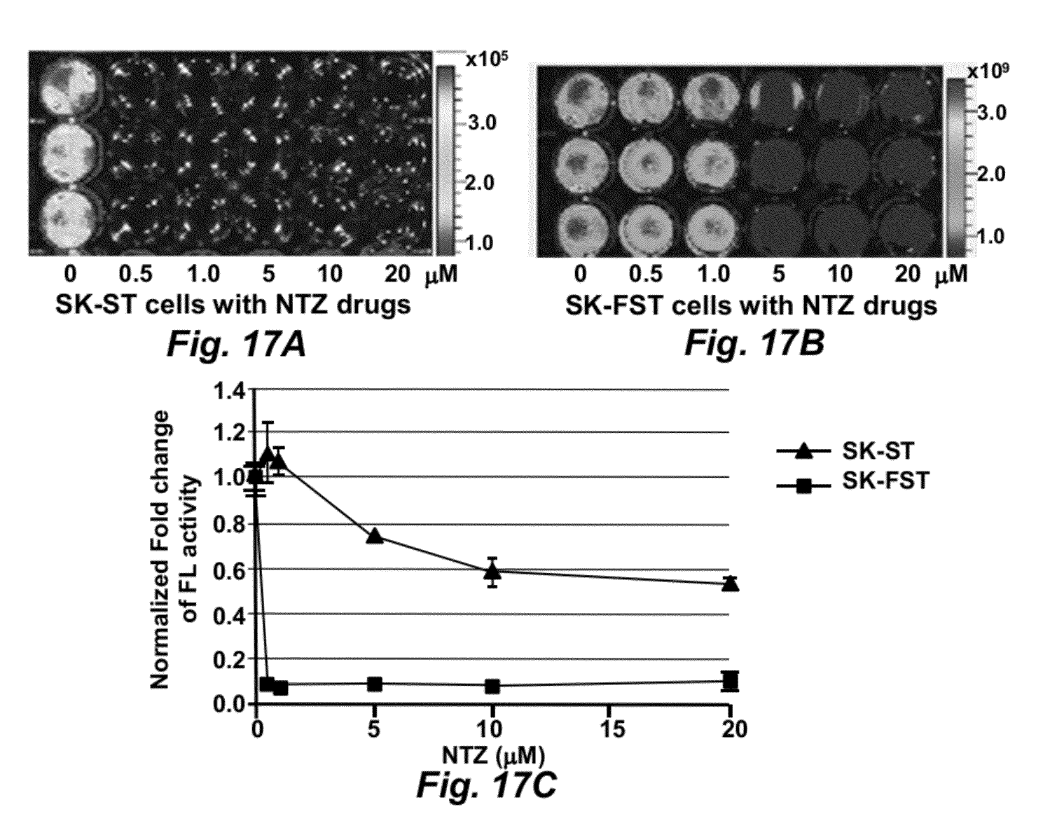

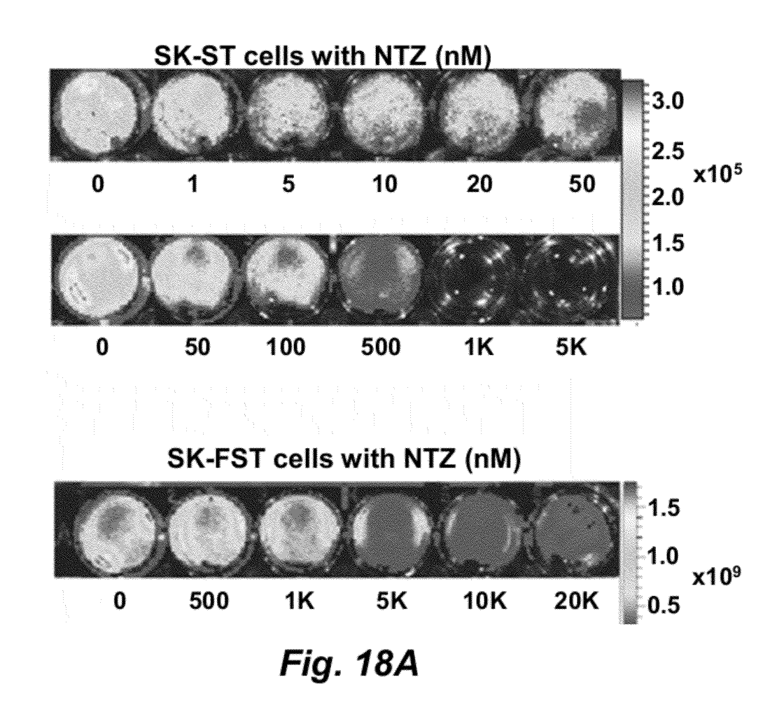

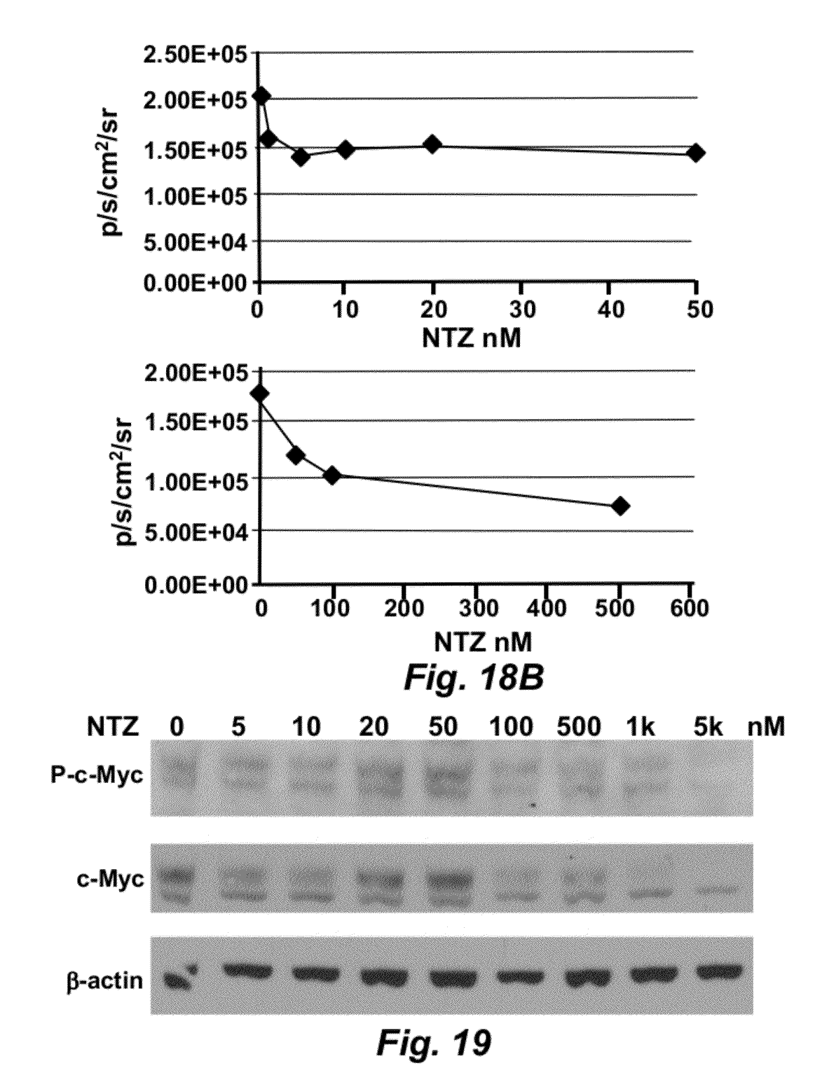

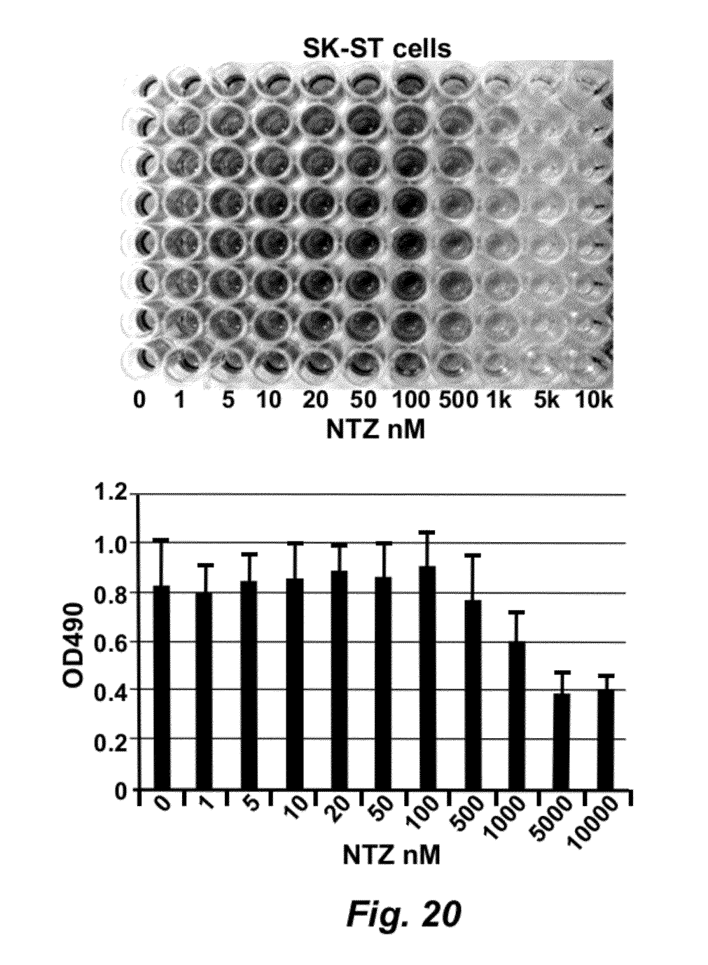

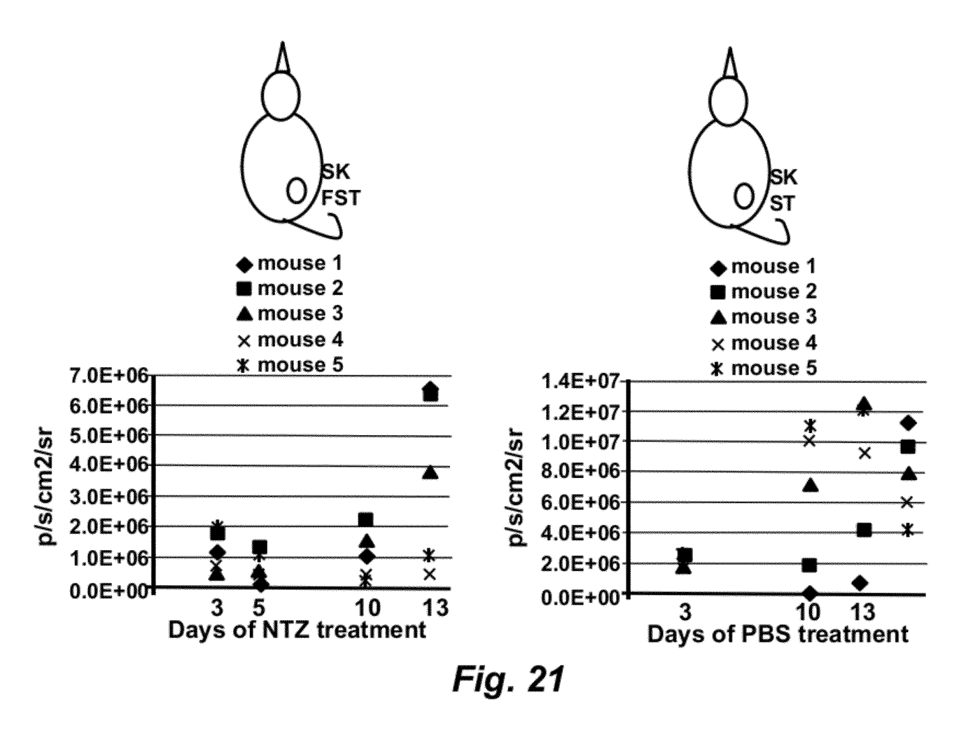

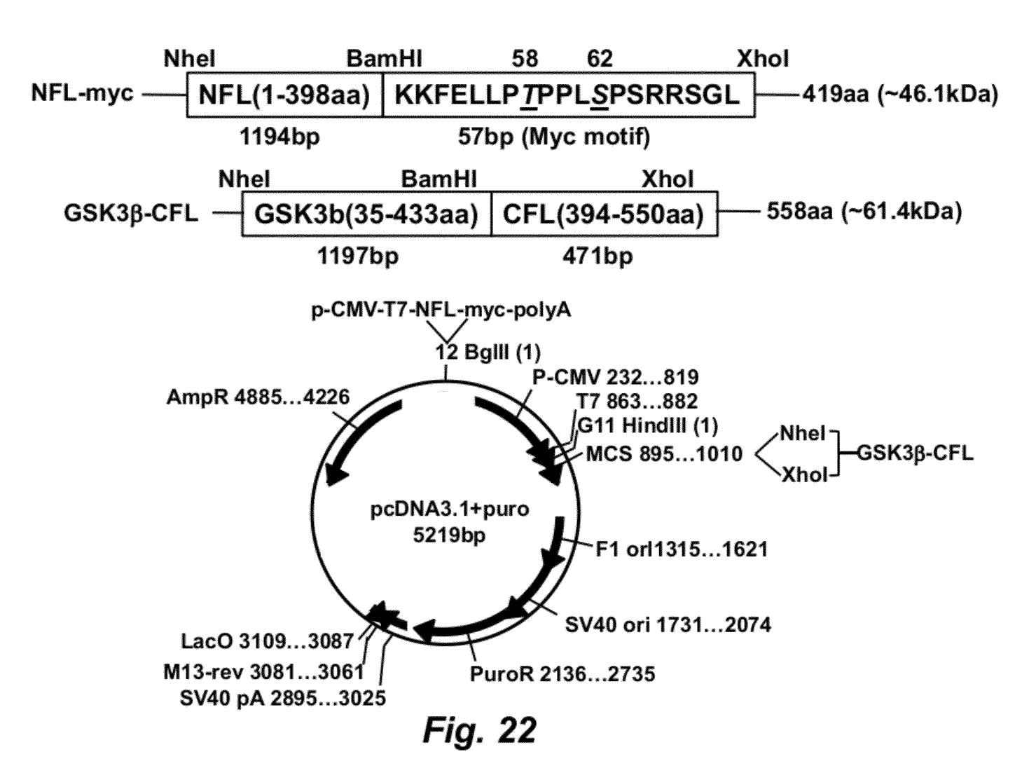

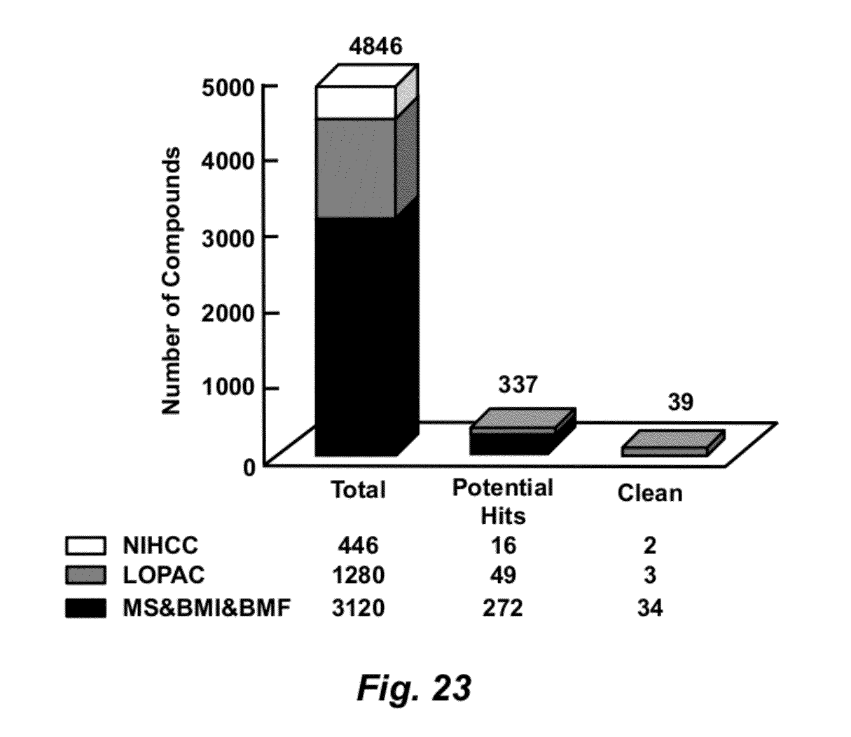

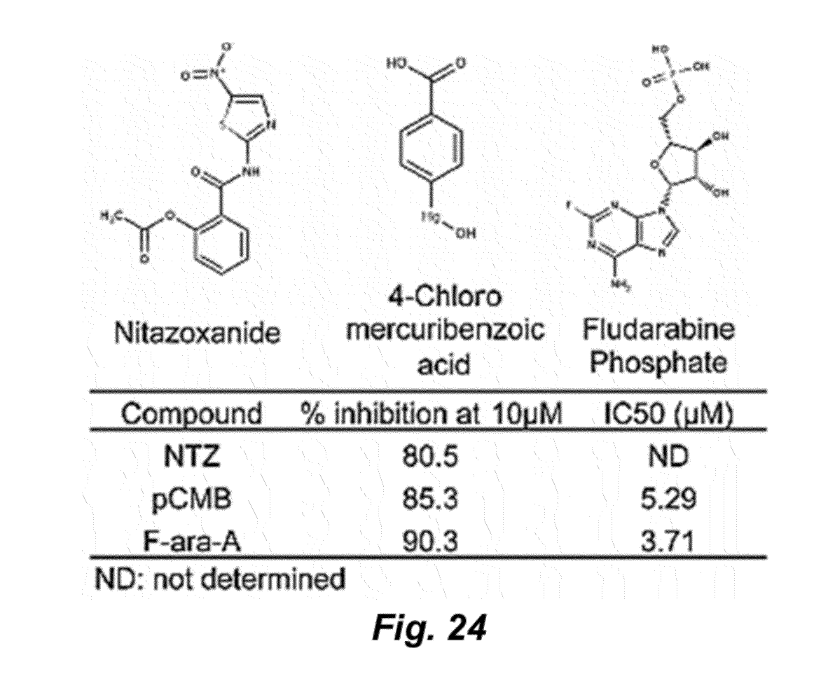

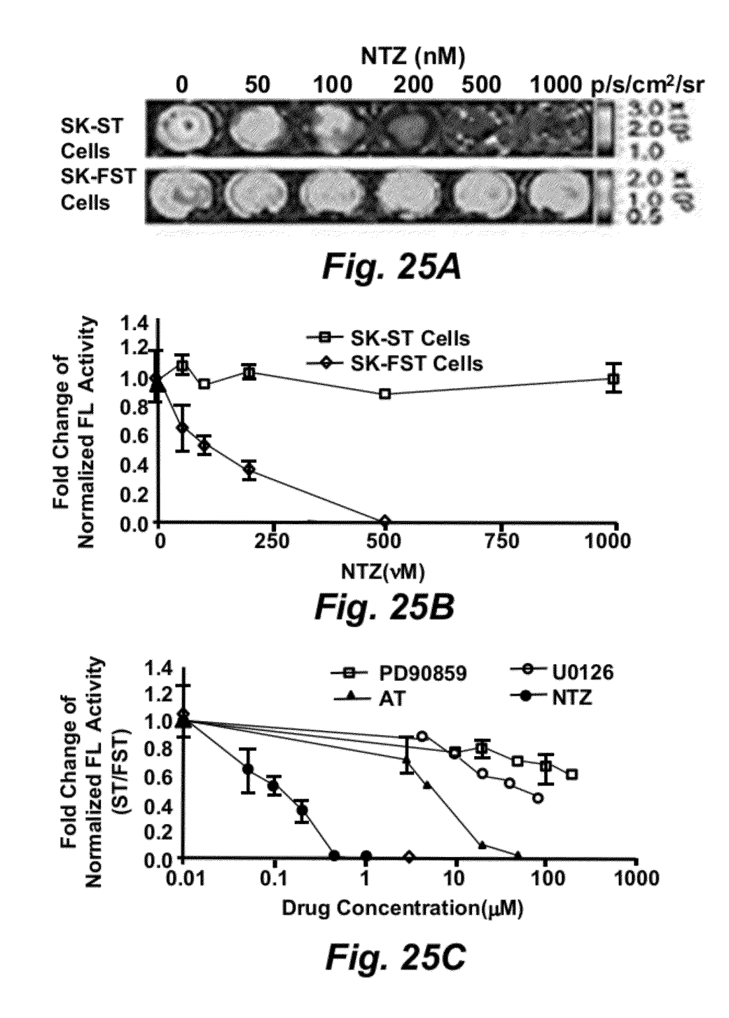

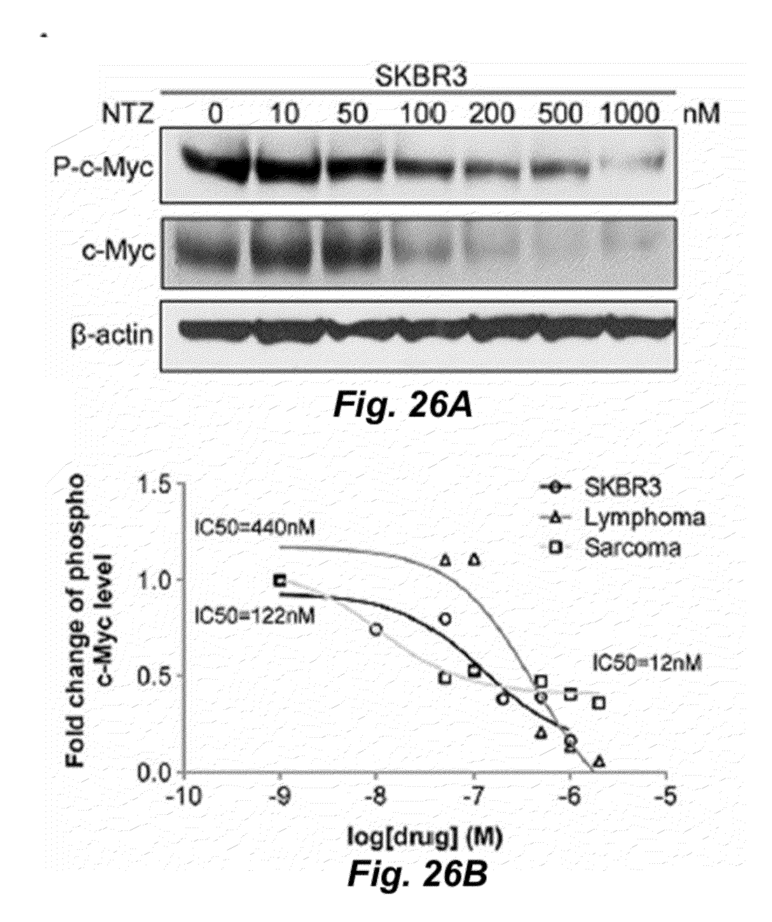

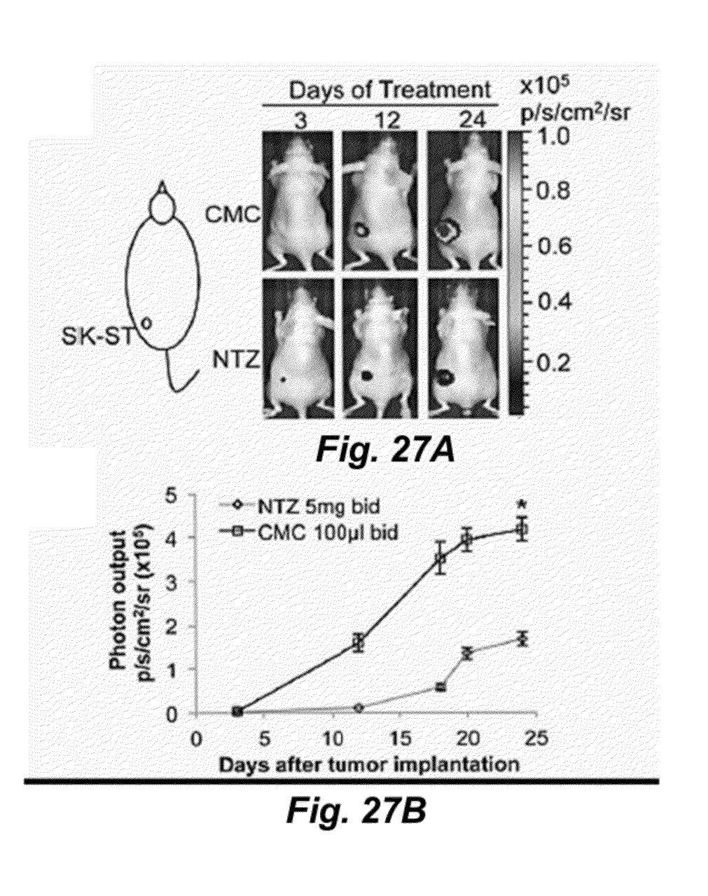

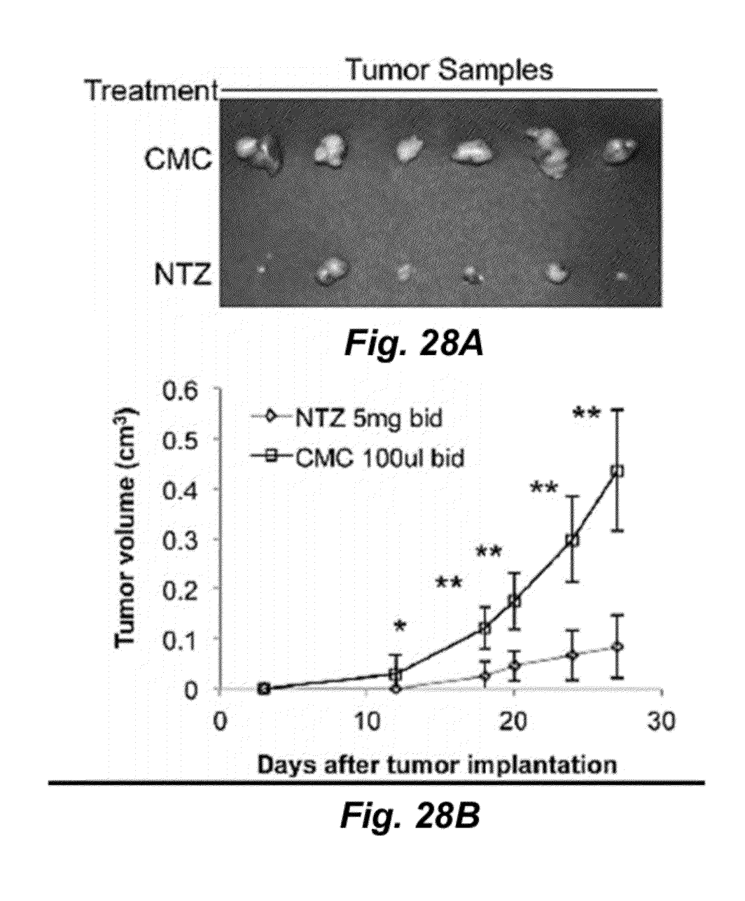

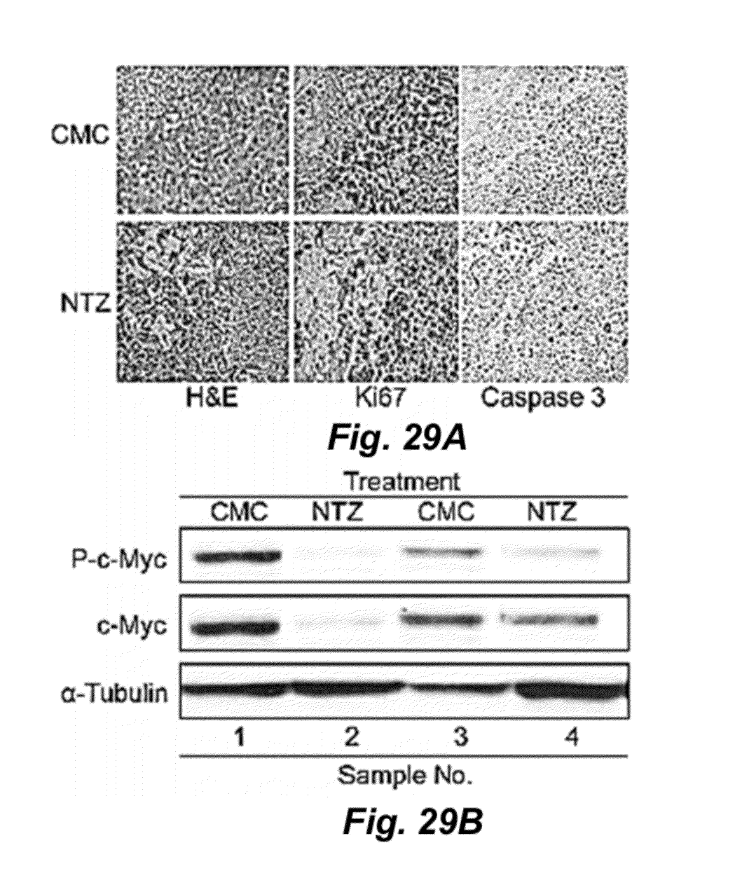

AT induced greater reduction in complemented FL activity with lower drug concentrations than did U0126, which in turn induced greater reduction than PD98059. These differential signal inhibitions also were correlated with the decreased endogenous c-Myc phosphorylation level, as shown in FIG. 6A. In 293T ST cells, however, AT induced a dose-dependent increase in complemented FL activity after normalization to its effect on the FL activity of 293T FST cells, which also was correlated with the increase in endogenous c-Myc phosphorylation level (FIGS. 6A, and 13A-13D). SKBR3 cells transfected with the mutant sensor GSK313/NFL-c-MycS62A were also subjected to PD98059 treatment, but did not respond to phosphorylation inhibition of c-Myc (FIGS. 14A and 14B).

Non-Invasive Imaging of c-Myc Phosphorylation in Mice.

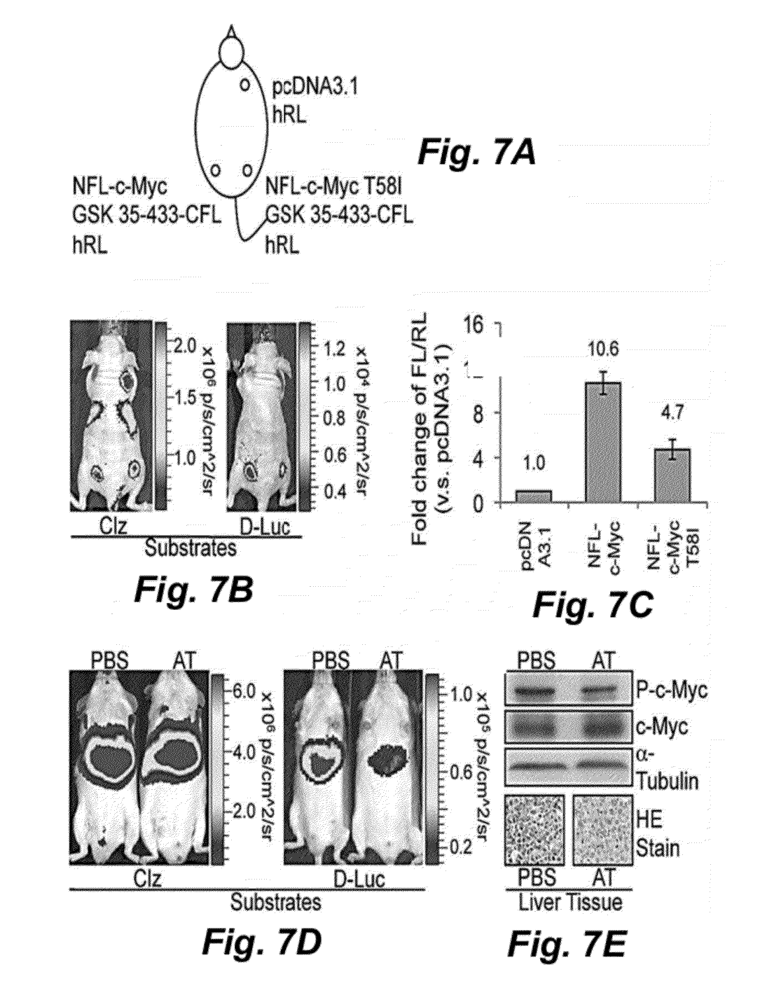

To test the in vivo application of the sensor system, SKBR3 cells were first transiently transfected separately with equal DNA molar amounts of the vectors pcDNA3.1(+), GSK 35-433-CFL/NFL-c-Myc or GSK 35-433-CFL/NFL-c-Myc T851 and implanted them subcutaneously in different locations in each nude mouse (n=3; FIG. 7A). hRL was also co-expressed for control of transfection efficiency. At 16 h after implantation, the three xenografts of each mouse all showed hRL activity on intravenous injection of its substrate coelenterazine (Clz) (FIG. 7B). However, on intraperitoneal injection of the FL substrate D-Luciferin (D-Luc), xenografts carrying pcDNA3.1(+) had very low complemented FL activity, whereas those carrying GSK 35-433-CFL/NFL-c-Myc had 10.6-fold higher activity. Those carrying GSK 35-433-CFL/NFL-c-Myc T851 had 4.7-fold higher activity after normalization to their hRL activity (FIG. 7C). Thus, the complemented FL activity determined by the phosphorylation status of NFL-c-Myc can be imaged and quantified in a mouse xenograft model. The sensor system was also transiently expressed in the liver of two transgenic mice using hydrodynamic injection. These mice carried a liver-specific transgene of c-Myc under control of the Tet system (Shachaf et al. (2007) Blood 110: 2674-2684).

Both mice had doxycycline discontinued, followed by a 29-day course of treatment with AT in one mouse and with PBS in the other mouse. After treatment, the two mice demonstrated no significant difference in liver tumor size. On the addition of Clz, both mice showed similar hRL activity, whereas on the addition of D-Luc, the mouse treated with AT had a much lower complemented FL activity than that treated with PBS (FIG. 7D). This signal reduction was consistent with the reduced endogenous c-Myc phosphorylation and the histological changes in the liver tissue samples (FIG. 7E).

In Vivo Monitoring of Drug Inhibition of c-Myc Phosphorylation

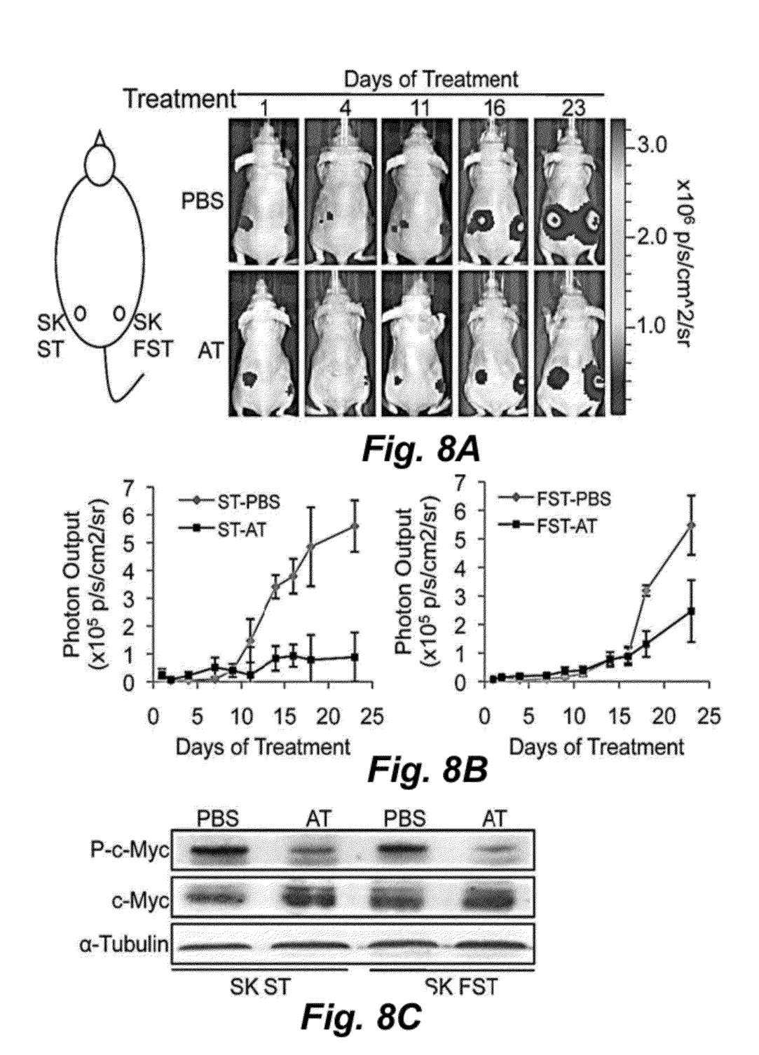

To further demonstrate the ability of the sensor system to monitor drug effects on c-Myc phosphorylation and tumor growth in vivo, SK ST cells were implanted subcutaneously to monitor phospho c-Myc levels and SK FST cells to control for cell numbers and the direct drug effect on FL activity in nude mice (n=10; FIG. 8A). The mice underwent gavage with AT (20 μL, 1.25 mM; n=5) or with PBS (20 μL; n=5) every other day. The complemented FL activity of SK ST xenografts began to rapidly increase 10 d after implantation in the PBS-treated group, but increased only slightly in the AT-treated group (FIG. 8B, left); yet there was no detectable difference in tumor size between the two groups. The FL activity of the SK FST xenografts increased gradually in both groups; up to about 18 d after implantation, FL activity was higher in the PBS-treated group compared with the AT-treated group (FIG. 8B, right). Normalization of the complemented FL activity of SK ST to that of SK FST showed a dramatic increase of sensor signal in early tumorigenesis in the PBS group, followed by a decrease due to the increase of tumor cell number. In the AT group, a sustained decrease in the sensor signal was seen (FIGS. 14A and 14B). Thus, the sensor detected the immediate c-Myc activity in tumorigenesis before tumor growth and thus non-invasively monitored the inhibitory effect of AT that eventually resulted in reduced tumor growth. The decreased sensor signal in the AT-treated group also was consistent with the decreased c-Myc phosphorylation levels in tumor tissue samples at 23 d after implantation (FIG. 8C).

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and the present disclosure and protected by the following claims.

Accordingly, one aspect of the present disclosure encompasses embodiments of a system for detecting the activation of a Myc peptide, the system comprising: a first polypeptide comprising a region isolated from a Myc polypeptide having at least one phosphor site associated with Myc activation and is resistant to ubiquitin-mediated proteosomal degradation, where said region is covalently linked to a first fragment of a luciferase; and a second polypeptide comprising a region of a glycogen synthase kinase 3β(GSK3β)capable of selectively interacting with a phosphorylated region of a Myc polypeptide, and a second fragment of the luciferase, where the region isolated from Myc polypeptide, when phosphorylated, can selectively bind to the glycogen synthase kinase 3β(GSK3β) region of the second polypeptide, allowing the first and the second luciferase fragments to cooperatively interact to produce a detectable signal.

In embodiments of this aspect of the disclosure, the region of a glycogen synthase kinase 3β (GSK3β) can extend from about amino acid position 35 to about amino acid position 433 of the amino acid sequence according to SEQ ID NO.: 8

In embodiments of this aspect of the disclosure, the region isolated from Myc polypeptide can comprise the sequence according to SEQ ID NO.: 1, or a conservative derivative thereof.

In embodiments of this aspect of the disclosure, the region of the glycogen synthase kinase 3β (GSK3β) polypeptide can comprise the sequence according to SEQ ID NO.: 2, or a conservative derivative thereof.

In embodiments of this aspect of the disclosure, the first polypeptide can have an amino acid sequence having at least 90% similarity to the amino acid sequence according to SEQ ID NO.: 9.

In embodiments of this aspect of the disclosure, the second polypeptide can have an amino acid sequence having at least 90% similarity to the amino acid sequence according to SEQ ID NO.: 10.

In embodiments of this aspect of the disclosure, the system can further comprise a genetically modified animal or human cell where the first and the second polypeptides can be expressed from at least one heterologous nucleic acid of the genetically modified animal or human cell.

In embodiments of this aspect of the disclosure, the genetically modified animal or human cell can respond to an exogenous ligand by phosphorylating the Myc polypeptide or region thereof, stimulating the association of the Myc polypeptide or region thereof and the glycogen synthase kinase 3β (GSK3β) region, allowing the first and the second fragments of the luciferase to cooperatively associate to generate a detectable signal.

Another aspect of the disclosure encompasses embodiments of a recombinant nucleic acid system comprising: (a) a first expression cassette comprising a first nucleotide sequence encoding a region of a Myc polypeptide resistant to ubiquitin-mediated proteosomal degradation and a first region of a luciferase, and where the first nucleotide sequence is operably linked to a promoter region for expressing the region of the Myc polypeptide and the first region of the luciferase as a single polypeptide; and (b) a second expression cassette comprising a second nucleotide sequence encoding a region of a glycogen synthase kinase 3β (GSK3β) and a second region of the luciferase, and where the second nucleotide sequence is operably linked to a second promoter region for expressing the region of a glycogen synthase kinase 3β (GSK3β) and the first region of the luciferase as a single polypeptide.

In embodiments of this aspect of the disclosure, the region of the glycogen synthase kinase 3β (GSK3β) can extend from amino acid position 35 to about position 433 of the amino acid sequence according to SEQ ID NO.: 8,

In embodiments of this aspect of the disclosure, the first and the second expression cassettes can each be in separate expression vectors, or are in the same expression vector.

In embodiments of this aspect of the disclosure, the region of a Myc polypeptide resistant to ubiquitin-mediated proteosomal degradation can be encoded by a nucleic acid sequence having at least 90% similarity to the sequence according to SEQ ID No.: 11; the first region of a luciferase can be encoded by a nucleotide sequence having at least 90% similarity to the sequence according to SEQ ID No.: 6, the region of a glycogen synthase kinase 3β (GSK3β) can be encoded by a nucleotide sequence having at least 90% similarity to the sequence according to SEQ ID No.: 3, and the second region of the luciferase can be encoded by a nucleotide sequence having at least 90% similarity to the sequence according to SEQ ID No.: 4.

In some embodiments of this aspect of the disclosure, the region of a Myc polypeptide resistant to ubiquitin-mediated proteosomal degradation can be encoded by a nucleic acid sequence according to SEQ ID No.: 11; the first region of a luciferase can be encoded by a nucleotide sequence according to SEQ ID No.: 6, the region of a glycogen synthase kinase 3β (GSK3β) can be encoded by a nucleotide sequence according to SEQ ID No.: 3, and the second region of the luciferase can be encoded by a nucleotide sequence according to SEQ ID No.: 4.

In embodiments of this aspect of the disclosure, the system can be within an animal or human cell.

Yet another aspect of the disclosure encompasses genetically modified animal or human cell or a population of genetically modified animal or human cells comprising a recombinant nucleic acid system according to any of the aforementioned embodiments.

Still another aspect of the disclosure encompasses embodiments of a method of detecting Myc activation in a population of animal or human cells, the method comprising the steps of: (i) providing a genetically modified population of animal or human cells comprising a recombinant nucleic acid system comprising: (a) a first expression cassette comprising a first nucleotide sequence encoding a region of a Myc polypeptide resistant to ubiquitin-mediated proteosomal degradation and a first region of a luciferase, and wherein the first nucleotide sequence is operably linked to a promoter region for expressing the region of the Myc polypeptide and the first region of the luciferase as a single polypeptide; and (b) a second expression cassette comprising a second nucleotide sequence encoding a region of a glycogen synthase kinase 3β (GSK3β) and a second region of the luciferase, and wherein the second nucleotide sequence is operably linked to a second promoter region for expressing the region of a glycogen synthase kinase 3β (GSK3β) and the first region of the luciferase as a single polypeptide; (ii) allowing the genetically modified population of animal or human cells to express the first and the second expression cassettes; (iii) contacting said cells with an agent characterized as stimulating the activation of a Myc polypeptide, thereby allowing the region of Myc polypeptide and the region of a glycogen synthase kinase 3β (GSK3β) of the expression products of the first and second cassettes to selectively bind to each other, and thereby allowing the first and the second fragments of the luciferase to cooperatively associate to produce a detectable signal; and (iv) detecting said signal, thereby detecting Myc activation in the cells.

In embodiments of this aspect of the disclosure, the method can further comprise the step of generating an image of the distribution of the signal in the cells.

In embodiments of this aspect of the disclosure, the genetically modified animal or human cell can be an in vitro cultured animal or human cell.

In embodiments of this aspect of the disclosure, the genetically modified population of animal or human cells can be in a recipient animal or human.

In embodiments of this aspect of the disclosure, the genetically modified population of animal or human cells can be in a recipient animal or human and the image of the distribution of the signal in the genetically modified population of animal or human cells further provides an image of the distribution of Myc activation in the animal or human.

In embodiments of this aspect of the disclosure, the method can further comprise the steps of: (v) detecting quantitatively a first signal, thereby determining a first level of Myc activation in the genetically modified population of animal or human cells; (vi) contacting the genetically modified population of animal or human cells with an agent suspected of modulating the activation of Myc and detecting quantitatively a second signal, thereby determining a second level of Myc activation in the genetically modified population of animal or human cells; and (vii) comparing the first and the second levels of Myc activation, thereby determining if the agent modulates Myc activation.

In embodiments of this aspect of the disclosure, the agent can selectively bind to a receptor of the genetically modified population of animal or human cells, thereby modulating a signaling pathway that activates Myc.

In embodiments of this aspect of the disclosure, the method can configured as a high-throughput assay system for the screening of a plurality of agents suspected of modulating Myc activation in a cell.

Yet another aspect of the disclosure encompasses embodiments of a method of inhibiting the activation of Myc by a cell, comprising contacting the cell with an effective amount of a nitazoxanide, or a derivative thereof, thereby reducing the activation of Myc.

In embodiments of this aspect of the disclosure, the cell can be a cancer cell and inhibiting the activation of Myc can reduce the proliferation of the cancer cell.

Still yet another aspect of the disclosure encompasses embodiments of a composition comprising a therapeutic dose of nitazoxanide or a derivative thereof, and a pharmaceutically acceptable carrier.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified.

### EXAMPLES

### Example 1

BLI of Living Mice: BLI of mouse xenografts was performed with the IVIS Spectrum optical imaging system (Xenogen) as described previously (Chan et al. (2008) Cancer Res. 68:216-226, incorporated herein by refrence in its entirety), with some modifications. At 4 h after transient transfection with the indicated constructs (FIG. 7A), 5×10⁶ SKBR3 cells were implanted subcutaneously into the indicated locations of nude mice (nu/nu; n=3). At 16 h after cell implantation, a Clz PBS (0.3 μg/μL; 100 μL) solution was injected intravenously for RL imaging. Then, 8 h later, FL imaging was acquired with D-Luc PBS solution (45 μg/μL; 100 μL) via intraperitoneal injection. Subsequently, 5×10⁶ each of SK ST and SK FST cells were implanted subcutaneously into nude mice (nu/nu; n=10) as indicated (FIG. 8A). FL imaging was performed 1 day after the implantation and every other day thereafter. After each FL imaging, half of the mice underwent gavage with AT PBS solution (1.25 mM; 20 μL), and the other half did so with PBS only (20 μL). After 23 d of treatment, the mice were killed, and xenograft tissue samples were collected for Western blot analysis of phospho c-Myc, c-Myc, and α-tubulin proteins.

### Example 2

Hydrodynamic Injection: Eμ-tTA/Tet-O-MYC transgenic mice (Shachaf et al. (2007) Blood 110: 2674-2684) (n=2) were removed from doxycycline treatment at 8 wk of age and underwent gavage with AT or PBS as described above. At 29 d after treatment, these two mice underwent imaging studies to characterize the liver phospho c-Myc level after hydrodynamic injection (Frame et al., (2001) Mol. Cell 7: 1321-1327) of the c-Myc sensor.

In brief, 5 μg of the single vector version of the sensor plasmid was mixed with 1 μg of hRL plasmid (as a control for transfection efficiency) into 2 mL of a 0.9% saline solution and injected within 8 s via the tail vein using a 3-mL syringe fitted with a 27-gauge needle. FL imaging was acquired with D-Luc at 22 h after injection. RL imaging was acquired with Clz at 48 h after injection.

The mice were killed after imaging, and liver tissue samples were collected for Western blot analysis of phospho c-Myc, c-Myc, and α-tubulin proteins and for H&E staining (Histo-Tec Laboratory).

### Example 3

Data Analysis: Curve fitting, correlation coefficients, and Student's t test were obtained using Microsoft Excel. Data are presented as mean±SD for in cellulo experiments and as mean±SEM for in vivo experiments. P<0.05 was used as cutoff point for statistical significance.

### Example 4

Plasmids: All constructs were built from donor plasmids FKBP12-Cfluc 394 and Nfluc 398-FRB, with a pcDNA3.1 (+) backbone and a CMV promoter. Different portions of the human GSK3β gene (FIG. 1B) were amplified by PCR from an existing clone and replaced the FKBP12 of FKBR12-Cfluc 394 as an NheI/BamHI fragment. The oligonucleotides for the 19 amino acid sequence KKFELLPTPPLSPSRRSGL (SEQ ID No.: 1) of the c-Myc activation motif replaced the FRB of Nfluc 398-FRB as a BamHI/XhoI fragment.

The corresponding split RL fusion constructs were built by replacing the Cfluc 394 and Nfluc 398 fragments with a BamHI/XhoI fragment of ChRL (230-311 aa) and a NheI/BamHI fragment of NhRL (1-229 aa), respectively, amplified by PCR from pCMV-hRL. Mutations at T58 and S62 were introduced into the NFL-c-Myc construct using the QUICK-CHANGE™ site-directed mutagenesis protocol (Stratagene).

For generation of stable cells, NFL-c-Myc was amplified as a BglII/BglII fragment containing both pCMV and polyA sequences and then inserted into the BglII site of GSK 35-433-CFL. The single construct carrying NFL-c-Myc and GSK 35-433-CFL genes with opposite transcription direction was used for the generation of stable cells. All sequences and mutations of fusion genes were confirmed by sequencing.

### Example 5

Cell Culture: All cell culture medium were supplemented with 10% FBS and 1% penicillin/streptomycin solution. CHO cells were cultured in F12 medium. SKBR3 (human breast adenocarcinoma) cells were cultured in McCoy's 5a medium. MCF-7 (human breast adenocarcinoma), HT1080 (fibrosarcoma), and 293T (human embryonic cancer) cells were cultured in DMEM medium. All cells were incubated at 37 ° C. with 5% CO₂. Transfection of CHO, SKBR3, MCF-7, and 293T cells were performed using LIPOFECTAMINE 2000™, as directed by the manufacturer. Transfection of HT1080 cells was performed using SUPERFECT™ (Qiagen), as directed by the manufacturer. SKBR3 and 293T stable cells were established by transient transfection of the sensor system on a single vector or the FL, and were selected with 3 μg/mL and 1 μg/mL of puromycin (Invitrogen), respectively.

### Example 6

Western Blot Analysis: Total cell lysates were collected using 1× cell lysis buffer (Cell Signaling), as directed by the manufacturer. The protein concentration was determined using the BioRad DC protein assay. SDS/PAGE analysis of 50 μg of protein was transferred to an lmmobilon-P membrane (Millipore). Anti-phosphor-c-Myc (Thr58/Ser62) and anti-c-Myc antibody (Cell Signaling) were used to visualize the phospho c-Myc and c-Myc protein, respectively, as directed by the manufacturer. Anti-FLuc-HRP antibody was used to detect the FL fragments, as directed by the manufacturer (Sigma-Aldrich). β-actin protein was used as a loading control and detected by anti-β-actin antibody (Sigma-Aldrich). Immunoblots were developed using the Pierce ECL Kit, as directed by the manufacturer. Protein levels were determined by quantifying the band intensities on Western blot analysis using ImageJ's ROI manager.

### Example 7

Selection of Fusion Fragments: The 51-69 aa in the MBI of c-Myc is highly conserved among higher species and different types of MYC proteins, consistent with a previously reported alignment (Cowling & Cole (2006) Semin. Cancer Biol. 16:242-252). This 19 amino acid sequence (SEQ ID No.: 1) is also part of the Myc transactivation domain that mediates c-Myc activation, but is not sufficient for ubiquitin-mediated proteasomal degradation (Muller & Eilers (2008) Ernst Schering Found. Symp. Proc. 2008: 99-113). To obtain a sufficient GSK3β sequence length for phospho recognition and kinase activity, different lengths of the protein were constructed based on its crystal structure (Dajani et al. (2001) Cell 105: 721-732.3). The fragment at 35-135 aa (GSK N7) contains the phospho recognition domain in a β-barrel structure formed by seven-stranded β sheets. The fragment at 35-350 aa (GSK 35-350) consists of both the phospho recognition domain and the kinase domain. Regions of 1-34 aa and 351-433 aa were unclear and disordered in the protein crystal and thus were considered to contain very flexible structures (3). The 1-34 aa region also contains Ser-9, the phosphorylation of which inhibits GSK3β kinase activity (Stambolic & Woodgett (1994) Biochem. J. 303: 701-704). Thus, fragment 35-433 aa (GSK 35-433) was deemed suitable for investigating the influence of the two flexible regions on the stability and specificity of the construct.

### Example 8

Luciferase Activity Assay: BLI of luciferase activity in intact cells was performed as described previously (Hann (2006) Semin Cancer Biol 16(4):288-302). In brief, 24 h after transfection, culture medium was replaced with a PBS solution (pH 7.0) of 45 μg/mL D-Luc for FL imaging or of 10 μg/mL of Clz for RL imaging with the IVIS BLI system (Xenogen). Images were acquired at 2-min intervals until the peak signal was reached. The photon output of FL or RL imaging in transiently transfected cells was normalized to co-expressed RL or FL activity, respectively, measured by an in vitro luminometry assay as described previously (Chan et al. (2008) Cancer Res. 68:216-226), then normalized to the total protein in the cell lysates, measured by the BioRad DC protein assay as directed by the manufacturer. In stable cells, the photon output of FL imaging was normalized to the total protein in the cell lysates.

### Example 9

Serum Stimulation Assay: CHO cells were co-transfected with the sensor system and hRL. After 24 h, cells were first serum-starved with 0.5% BSA F12 medium for 18 h, then serum-stimulated with 20% FBS F12 medium and imaged at the indicated time points (FIGS. 5A-5C) with D-Luc in the IVIS 50 system. The photon output of FL imaging was normalized to hRL activity and the total protein content, measured as in the luciferase activity assay. CHO cells were treated as above and lysed at the indicated time points for Western blot analysis of phospho c-Myc, c-Myc, and β-actin proteins. The correlation coefficient between the fold change of normalized FL activity and that of normalized phospho c-Myc level (phospho c-Myc/β-actin) was determined based on the R2 value.

### Example 10

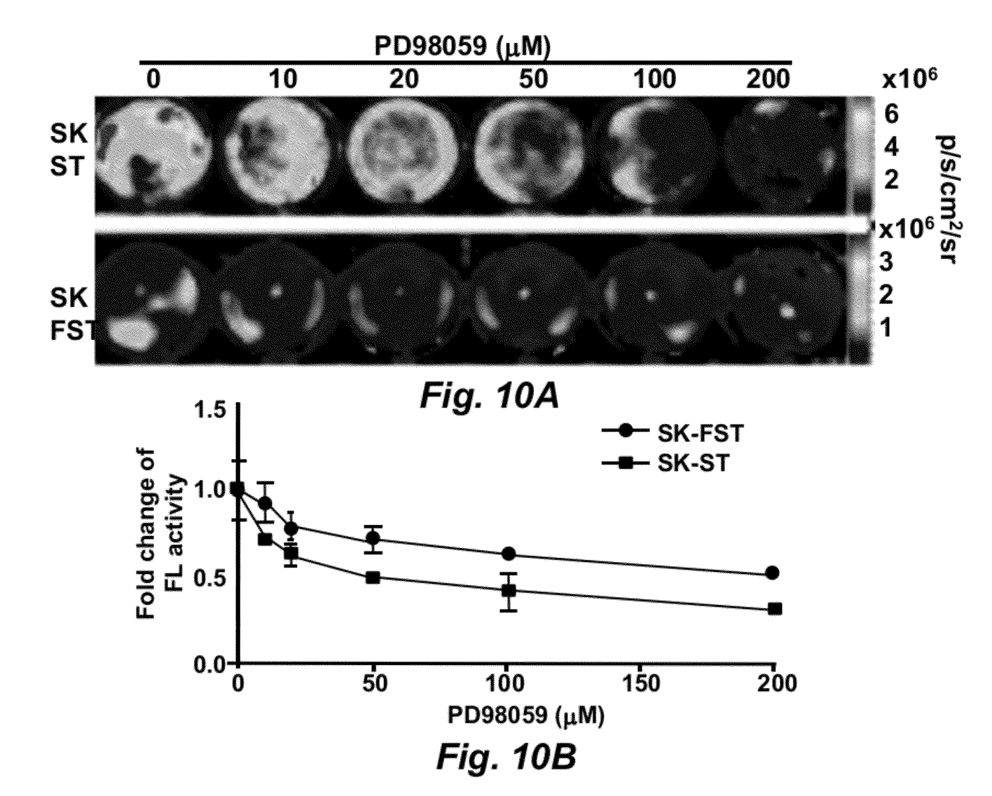

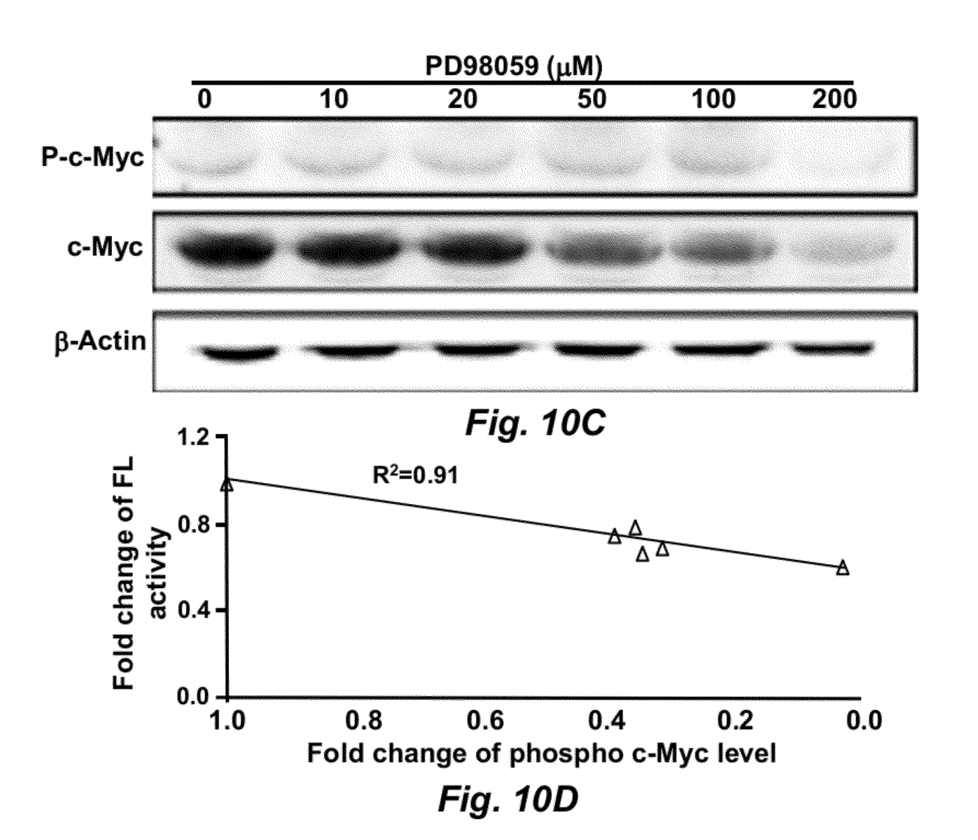

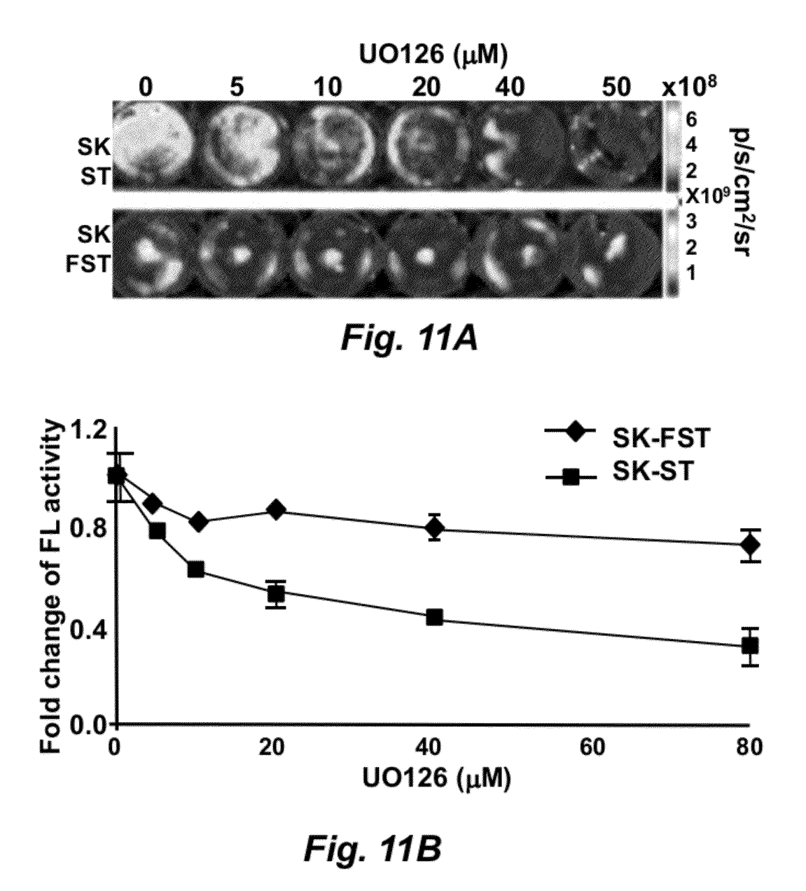

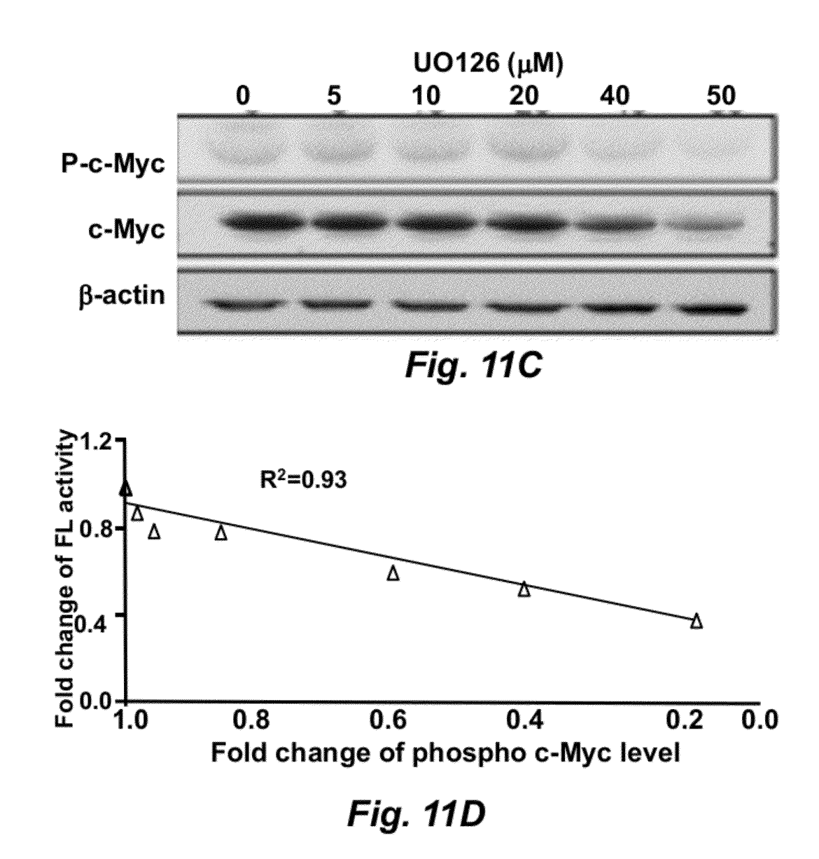

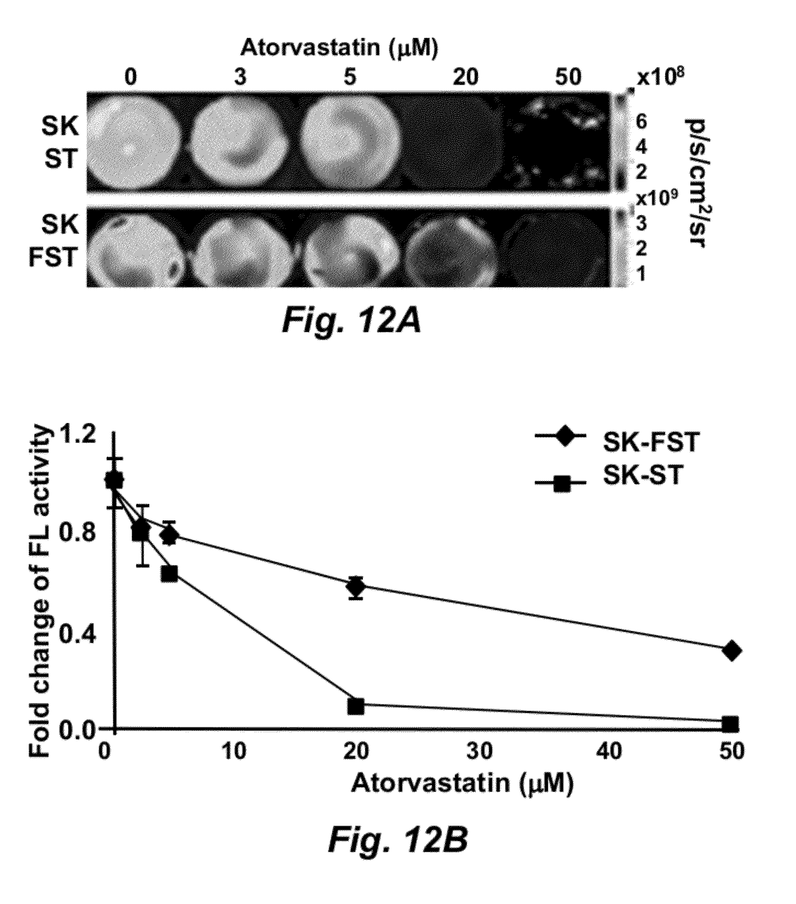

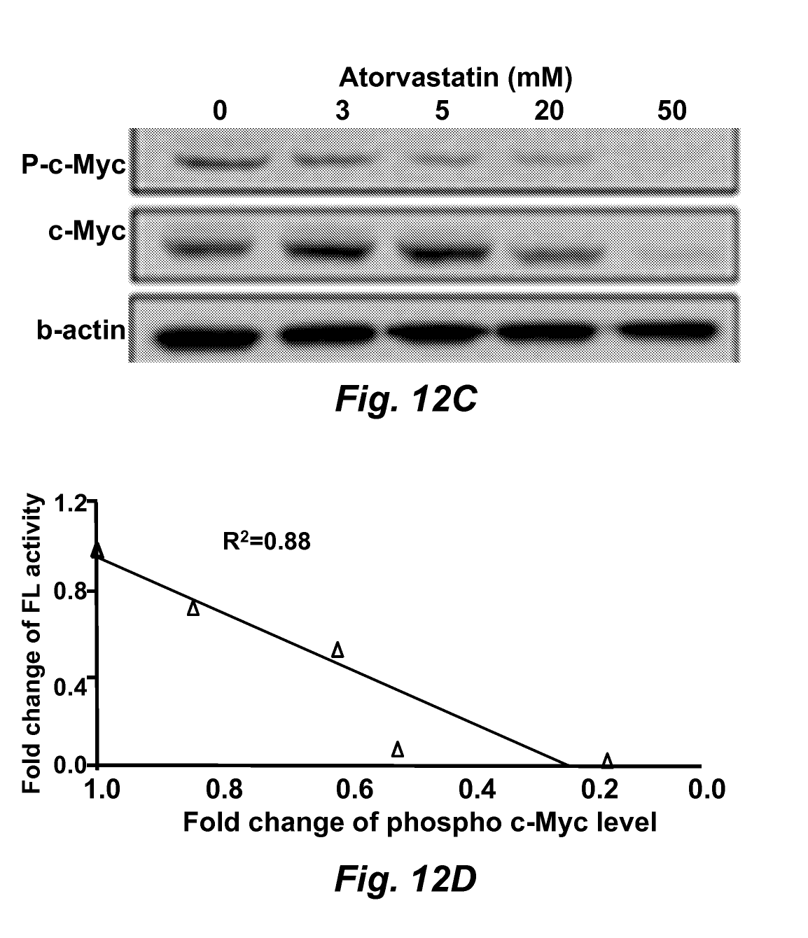

Drug Inhibition Assay: SKBR3 stable cells constitutively expressing the sensor system (SK ST) or the full-length FL (SK FST) were treated with PD98059 (Cell Signaling) for 2 h, U0126 (Cell Signaling) for 1 h, and purified AT (Sequoia Research Products) for 16 h at the indicated concentrations and imaged with D-Luc in the IVIS 50 BLI system (FIGS. 6A and 6B, and FIGS. 10A-12D). In addition, 293T stable cells constitutively expressing the sensor system (293T ST) or the full-length FL (293T FST) were treated with AT for 16 h at the indicated concentrations (FIGS. 6A and 6B, and FIGS. 13A-13D) and imaged with D-Luc in the IVIS 50 system. The photon output of FL imaging was normalized to the total protein content, and the FL activity of SK ST or 293T ST was normalized to that of SK FST or 293T FST, respectively. SKBR3 and 293T cells were treated as above and lysed for Western blot analysis of phospho c-Myc, c-Myc, and β-actin proteins. The correlation coefficient between the fold change of normalized FL activity and the fold change of normalized phospho c-Myc level (phospho c-Myc/β-actin) was determined based on the R² value.

1. A system for detecting the activation of a Myc peptide, the system comprising:
a first polypeptide comprising a region isolated from a Myc polypeptide having at least one phosphor site associated with Myc activation and is resistant to ubiquitin-mediated proteosomal degradation, wherein said region is covalently linked to a first fragment of a luciferase; and
a second polypeptide comprising a region of a glycogen synthase kinase 36 (GSK3β) capable of selectively interacting with a phosphorylated region of a Myc polypeptide, and a second fragment of the luciferase,
wherein the region isolated from Myc polypeptide, when phosphorylated, selectively binds to the glycogen synthase kinase 3β (GSK3β) region of the second polypeptide, thereby allowing the first and the second luciferase fragments to cooperatively interact to produce a detectable signal.

2. The system according to claim 1, wherein the region of a glycogen synthase kinase 3β (GSK3β) extends from about amino acid position 35 to about amino acid position 433 of the amino acid sequence according to SEQ ID NO.: 8.

3. The system according to claim 1, wherein the region isolated from Myc polypeptide comprises the sequence according to SEQ ID NO.: 1, or a conservative derivative thereof.

4. The system according to claim 1, wherein the region of the glycogen synthase kinase 3β (GSK3β) polypeptide comprises the sequence according to SEQ ID NO.: 2, or a conservative derivative thereof.

5. The system according to claim 1, wherein the first polypeptide has an amino acid sequence having at least 90% similarity to the amino acid sequence according to SEQ ID NO.: 9.

6. The system according to claim 1, wherein the second polypeptide has an amino acid sequence having at least 90% similarity to the amino acid sequence according to SEQ ID NO.: 10.

7. The system according to claim 1, further comprising a genetically modified animal or human cell wherein the first and the second polypeptides are expressed from at least one heterologous nucleic acid of the genetically modified animal or human cell.

8. The system according to claim 7, wherein the genetically modified animal or human cell can respond to an exogenous ligand by phosphorylating the Myc polypeptide or region thereof, thereby stimulating the association of the Myc polypeptide or region thereof and the glycogen synthase kinase 3β (GSK3β) region, thereby allowing the first and the second fragments of the luciferase to cooperatively associate to generate a detectable signal.

9. A recombinant nucleic acid system comprising:
(a) a first expression cassette comprising a first nucleotide sequence encoding a region of a Myc polypeptide resistant to ubiquitin-mediated proteosomal degradation and a first region of a luciferase, and wherein the first nucleotide sequence is operably linked to a promoter region for expressing the region of the Myc polypeptide and the first region of the luciferase as a single polypeptide; and
(b) a second expression cassette comprising a second nucleotide sequence encoding a region of a glycogen synthase kinase 3β (GSK3β) and a second region of the luciferase, and wherein the second nucleotide sequence is operably linked to a second promoter region for expressing the region of a glycogen synthase kinase 3β (GSK3β) and the first region of the luciferase as a single polypeptide.

10. The recombinant nucleic acid system according to claim 9, wherein the region of the glycogen synthase kinase 3β (GSK3β) extends from amino acid position 35 to about position 433 of the amino acid sequence according to SEQ ID NO.: 8,

11. The recombinant nucleic acid system according to claim 9, wherein the first and the second expression cassettes are each in separate expression vectors, or are in the same expression vector.

12. The recombinant nucleic acid system according to claim 9, wherein the region of a Myc polypeptide resistant to ubiquitin-mediated proteosomal degradation is encoded by a nucleic acid sequence having at least 90% similarity to the sequence according to SEQ ID No.: 11; the first region of a luciferase is encoded by a nucleotide sequence having at least 90% similarity to the sequence according to SEQ ID No.: 6, and wherein the region of a glycogen synthase kinase 3β (GSK3β) is encoded by a nucleotide sequence having at least 90% similarity to the sequence according to SEQ ID No.: 3, and the second region of the luciferase is encoded by a nucleotide sequence having at least 90% similarity to the sequence according to SEQ ID No.: 4.

13. The recombinant nucleic acid system according to claim 9, wherein the region of a Myc polypeptide resistant to ubiquitin-mediated proteosomal degradation is encoded by a nucleic acid sequence according to SEQ ID No.: 11; the first region of a luciferase is encoded by a nucleotide sequence according to SEQ ID No.: 6, the region of a glycogen synthase kinase 3β (GSK3β) is encoded by a nucleotide sequence according to SEQ ID No.: 3, and the second region of the luciferase is encoded by a nucleotide sequence according to SEQ ID No.: 4.

14. The recombinant nucleic acid system according to claim 11, wherein said system is within an animal or human cell.

15. A genetically modified animal or human cell or a population of genetically modified animal or human cells comprising a recombinant nucleic acid system according to claim 9.

16. A method of detecting Myc activation in a population of animal or human cells, the method comprising the steps of:
(i) providing a genetically modified population of animal or human cells comprising a recombinant nucleic acid system comprising:
(a) a first expression cassette comprising a first nucleotide sequence encoding a region of a Myc polypeptide resistant to ubiquitin-mediated proteosomal degradation and a first region of a luciferase, and wherein the first nucleotide sequence is operably linked to a promoter region for expressing the region of the Myc polypeptide and the first region of the luciferase as a single polypeptide; and
(b) a second expression cassette comprising a second nucleotide sequence encoding a region of a glycogen synthase kinase 3β (GSK3β) and a second region of the luciferase, and wherein the second nucleotide sequence is operably linked to a second promoter region for expressing the region of a glycogen synthase kinase 3β (GSK3β) and the first region of the luciferase as a single polypeptide;
(ii) allowing the genetically modified population of animal or human cells to express the first and the second expression cassettes;
(iii) contacting said cells with an agent characterized as stimulating the activation of a Myc polypeptide, thereby allowing the region of Myc polypeptide and the region of a glycogen synthase kinase 3β (GSK3β) of the expression products of the first and second cassettes to selectively bind to each other, and thereby allowing the first and the second fragments of the luciferase to cooperatively associate to produce a detectable signal; and
(iv) detecting said signal, thereby detecting Myc activation in the cells.

17. The method of claim 16, further comprising the step of generating an image of the distribution of the signal in the cells.

18. The method of claim 16, wherein the genetically modified animal or human cell is an in vitro cultured animal or human cell.

19. The method of claim 16, wherein the genetically modified population of animal or human cells is in a recipient animal or human.

20. The method of claim 16, wherein the genetically modified population of animal or human cells is in a recipient animal or human and the image of the distribution of the signal in the genetically modified population of animal or human cells further provides an image of the distribution of Myc activation in the animal or human.

21. The method of claim 16, further comprising the steps of:
(v) detecting quantitatively a first signal, thereby determining a first level of Myc activation in the genetically modified population of animal or human cells;
(vi) contacting the genetically modified population of animal or human cells with a agent suspected of modulating the activation of Myc and detecting quantitatively a second signal, thereby determining a second level of Myc activation in the genetically modified population of animal or human cells; and
(vii) comparing the first and the second levels of Myc activation, thereby determining if the agent modulates Myc activation.

22. The method of claim 21, wherein the agent selectively binds to a receptor of the genetically modified population of animal or human cells, thereby modulating a signaling pathway that activates Myc.

23. The method of claim 16, wherein the method is configured as a high-throughput assay system for the screening of a plurality of agents suspected of modulating Myc activation in a cell.

24. A method of inhibiting the activation of Myc by a cell, comprising contacting the cell with an effective amount of a nitazoxanide, or a derivative thereof, thereby reducing the activation of Myc.

25. The method of claim 24, wherein the cell is a cancer cell and inhibiting the activation of Myc reduces the proliferation of the cancer cell.

26. A composition comprising a therapeutic dose of nitazoxanide or a derivative thereof, and a pharmaceutically acceptable carrier.
